(19) United States
(12) Patent Application Publication
(10) Pub. No.: US 2020/0163334 A1
(43) Pub. Date: May 28, 2020

(54) INSECT REPELLENT

(71) Applicant: FMC Corporation, Philadelphia, PA (US)

(72) Inventors: Robert B. Albright, Lansdale, PA (US); Dina L. Richman, Philadelphia, PA (US); Nathan D. Caldwell, Morrisville, PA (US); Bruce C. Black, Yardley, PA (US)

(21) Appl. No.: 16/080,434

(22) PCT Filed: Feb. 28, 2017
(86) PCT No.: PCT/US17/19832

§ 371 Date: Aug. 28, 2018

Related U.S. Application Data
(60) Provisional application No. 62/301,364, filed on Feb. 29, 2016.

(51) Int. Cl. A01N 37/44; A01N 25/34; A61K 8/36; A61Q 17/04

(52) U.S. Cl. CPC A01N 37/44; A61Q 17/04; A61K 8/361; A01N 25/34

(57) ABSTRACT

In one aspect, this invention relates to pest repellent compositions comprising a fatty acid and an anthranilate ester. In another aspect, this invention relates to a method of repelling pests employing such composition. In another aspect, this invention relates to products formulated from such composition.

### CROSS REFERENCE TO RELATED APPLICATIONS

This application claims the benefit of priority of U.S. Provisional Application No. 62/301,364, filed on Feb. 29, 2016, which is incorporated by reference in its entirety.

### FIELD OF THE INVENTION

In one aspect, this invention relates to pest repellent composition comprising a fatty acid and an anthranilate ester. In another aspect, this invention relates to a method of repelling pests employing such composition.

### BACKGROUND OF THE INVENTION

Insect and acarid pests can cause severe damage to crops and horticultural plants. Moreover insects and pests can carry agents that cause disease in both people and animals. For example, mosquitoes can carry viruses that cause encephalitis, and ticks can carry bacteria that cause Lyme disease. One method of controlling such pests involves the application of chemicals which repels such pests from a given environment. Thus, chemicals such as N,N-diethyl-m-toluamide (also known as DEET) is employed in repellents to protect individuals from mosquitoes, ticks and other similar pests. Other chemicals which affect insect behavior are used to attract or repel insects from a given environment in order to enhance the effectiveness of insecticides, either by attracting insects to the area where insecticides can be more effectively employed, or by repelling them from areas where insecticides are inefficient.

Among the compounds which have been employed to influence insect behavior so as to cause them to move to a different environment is methyl anthranilate. Thus, U.S. Pat. No. 6,958,146 (Askham et al), U.S. Pat. No. 7,867,479 (Dunham et al) and U.S. Pat. No. 8,092,790 (Dunham et al) all disclose the use of methyl anthranilate in the form of the commercial product BIRDSHIELD™ to induce insects to migrate from one environment to another (see, for example. Column 9, lines 26-33 of U.S. Pat. No. 7,867,479).

Anthranilate esters such as methyl anthranilate have long been known to be useful as bird repellents. Thus. U.S. Pat. No. 2,967,128 (Kare) describes the use of such compounds to deter both domestic and wild birds from eating seeds, berries, grains, fruits and the like. Further, such compounds have found to be insect attractants—for example, U.S. Pat. No. 5,296,226 (Askham) states (at Column 3, lines 20-23) that “insects are readily attracted to dimethyl and methyl anthanilate. Crops relatively free of insects were quickly reinfested after being treated with either material.” This finding is supported by the disclosures in U.S. Pat. No. 6,958,146 (Askham et al), U.S. Pat. No. 7,867,479 (Dunham et al) and U.S. Pat. No. 8,092,790 (Dunham et al) which show in Table 1 of such publications that sticky traps containing methyl anthranilate quickly became covered with hundreds of insects. The sole exception presented in these patents are house flies (Musca domesticae) which were repelled by the use of methyl anthranilate.

Although U.S. Pat. Nos. 6,958,146, 7,867,479 and 8,092,790 all describe BIRDSHIELD™ as being a mixture of methyl anthranilate with a fatty acid, it is noted that the label for such product indicates that it is covered by the claims of U.S. Pat. No. 5,296,226. This patent is directed to bird repellant compositions comprising an anionic surfactant consisting of an alkyl metal salt of a fatty acid; rather than a fatty acid in acid form. According to this publication, the addition of such fatty acid salts results in the formation of micelles of such anthranilate compounds, permitting a more even distribution of such compounds on the surface treated and enhancing their efficacy as bird repellents.

Given that compositions comprising an anthranilate ester and a fatty acid salt will attract most insects and will repel birds, it is completely unexpected that a composition comprising an anthranilate ester and a fatty acid in acid form will effectively repel many insect species as well as acarids, without repelling birds, making them useful for a number of repellent purposes including protecting bird feeders from unwanted insects.

Furthermore, many substances are applied topically to the skin or mucous membranes of humans or animals (hereafter “skin”) in order to alter the subject's appearance, to protect the subject from the environment, or to produce a biological change in the skin or other tissue for therapeutic, preventive or cosmetic purposes. These substances may generically be termed “consumer care products” and include such topically applied substances as cosmetics, over-the-counter and prescription topical drugs, and a variety of other products such as soaps and detergents.

Consumer care products occur in a variety of forms, including solids, liquids, suspensions, semisolids (such as creams, gels, pastes or “sticks”), powders or finely dispersed liquids such as sprays or mists. Examples of consumer care products commonly classified as “cosmetics” include skin care products such as creams, lotions, moisturizers and “treatment cosmetics” such as exfoliants and/or skin cell renewal agents; fragrances such as perfumes and colognes, and deodorants; shaving-related products such as creams, “bracers” and aftershaves; depilatories and other hair removal products; skin cleansers, toners and astringents; pre-moistened wipes and washcloths; tanning lotions; bath products such as oils; eye care products such as eye lotions and makeup removers; foot care products such as powders and sprays; skin colorant and make-up products such as foundations, blushes, rouges, eye shadows and liners, lip colors and mascaras; lip balms and sticks; hair care and treatment products such as shampoos, conditioners, colorants, dyes, bleaches, straighteners and permanent wave products; baby products such as baby lotions, oils, shampoos, powders and wet wipes; feminine hygiene products such as deodorants and douches; skin or facial peels applied by dermatologists or cosmeticians; and others.

Examples of consumer care products commonly classified as “topical drugs” are many and varied, and include over-the-counter and/or prescription products such as antiperspirants, insect repellents, sunscreens and sunburn treatments, anti-acne agents, antibiotics, topical respiratory agents, ocular drugs such as eyedrops and saline solutions, therapeutic retinoids, anti-dandruff agents, external analgesics such as capsaicin products, topical contraceptives, topical drug delivery systems, gastrointestinal agents such as suppositories, enemas and hemorrhoid treatments, reproductive system agents such as vaginal treatments, oral treatments such as lozenges, and many other products with therapeutic or other effects. Other consumer care products include hand, facial and body soaps and detergents and other forms of skin cleansers, as well as household detergents and many other household products such as solvents, propellants, polishes, lubricants, adhesives, waxes and others which are either applied topically or are topically exposed to the body during normal use.

Applicants have recognized the need to develop consumer care products containing an anthranilate ester and a fatty acid salt that can effectively repel many insect species as well as acarids, without repelling birds, making them useful for a number of repellent purposes including protecting bird feeders from unwanted insects.

### SUMMARY OF THE INVENTION

In one aspect, the present invention is directed to a composition comprising an insect, or acarid repellant amount of a composition comprising: (a) an anthranilate ester; and (b) a fatty acid.

In another aspect this invention is directed to a method of repelling insect and/or acarid pests employing such composition.

In another embodiment, this invention relates to consumer care products for repelling insects and/or acarid pests.

In another aspect, the present invention is directed to a composition comprising an insect or acarid repellant comprising: (a) a first active ingredient selected from the group consisting of dimethyl anthranilate, methyl anthranilate, ethyl anthranilate, phenylethyl anthranilate and menthyl anthranilate; (b) a fatty acid; (c) optionally at least one additional active ingredient and (c) a pharmaceutically suitable topical carrier; wherein the ratio of first active ingredient to fatty acid is about 6:1; 5:1; 4:1; or 3:1. In the preferred embodiment this ration is about 5:1 to 4:1.

Another aspect this invention is directed to the composition comprising an insect or acarid repellant described herein, where the fatty acid is selected from the group consisting of oleic acid, ricinoleic acid, linoleic acid palmitic acid, stearic acid, and any mixtures thereof.

Another aspect this invention is directed to a composition comprising an insect or acarid repellant described herein, wherein the fatty acid is oleic acid.

Another aspect this invention is directed to a composition comprising an insect or acarid repellant described herein, wherein the active ingredient is an anthranilate ester selected from the group consisting of dimethyl anthranilate, methyl anthranilate, ethyl anthranilate, phenylethyl anthranilate and menthyl anthranilate. In certain embodiments, the anthranilate ester is methyl anthranilate.

Another aspect this invention is directed to a composition comprising an insect or acarid repellant described herein, further comprising an additional compound selected from the group consisting of a surfactant, emulsifying agent, a pH modifier, a thickening agent, an anti-oxidant, a preservative, a skin conditioning agent, an emollient, a humectant, a silicone moiety, an anti-inflammatory, a sun blocking agent, a topical anesthetic, vitamins, an antipruritic, an antibiotic, antifungal, an antiseptic, a vasoconstrictor, and any mixtures thereof.

In certain embodiments, the emulsifying agent is selected from the group consisting of polysaccharide ethers, polyglycosides, fatty acids, fatty alcohols, amine oxides, water-soluble cellulose, alkyl sulfonates, ethoxylated alkyl phenols, alkanolamides, betaines, zwitterionic surfactants, carboxylated alcohols, carboxylic acids, ethoxylated C12-C15 alcohols, polysorbates, behentrimonium methosulfate, isopropyl myristate and ethoxylated castor oil or any mixtures thereof.

In certain embodiments, the thickening agent is selected from the group consisting of carboxylic acid polymers, crosslinked acrylic acid with allyl ethers of sucrose, crosslinked polyacrylate polymers, polyacrylamide polymers, polysaccharides, gums and mixtures thereof.

In certain embodiments, the polysaccharides is selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropylmethylcellulose, methyl hydroxyethylcellulose, microcrystalline cellulose, sodium cellulose sulfate, chitosan and mixtures thereof.

In certain embodiments, the gum is selected from the group consisting of calcium carrageenan, carnitine, carrageenan, dextrin, gelatin, gellan gum, guar gum, guar hydroxypropyltrimonium chloride, hectorite, hydroxypropyl guar, karaya gum, kelp, locust bean gum, natto gum, potassium carrageenan, sclerotium gum, sodium carrageenan, tragacanth gum, xanthan gum and any mixtures there of.

Another aspect this invention is directed to a composition comprising an insect or acarid repellant described herein, wherein the pharmaceutically suitable topical carrier comprise water, glycerin, lecithin a fatty acid or any combinations thereof.

In certain embodiments, the fatty acid is selected from the group consisting of oleic acid, ricinoleci acid, linoleic acid, palmitic acid steric acid or any mixtures thereof.

In certain embodiments, the sun blocking agent is selected from the group consisting of PABA, PABA esters, butyl PABA, ethyl PABA, ethyl dihydroxypropyl PABA, benzophenones, avobenzone, cinnamates, salicylates anthranilates, ethyl urocanate, homosalate, dibenzoylmethanes, octocrylene, methylbenzylidene camphor, kaolin, talc, petrolatum and metal oxides or any mixtures thereof.

Other aspects of this invention include a method of repelling an insect from a subject comprising topically administering to a subject in need thereof the composition comprising an insect or acarid repellant described herein.

In certain embodiments, the subject is a mammalian subject. In other embodiments, the subject is a human subject. In other embodiments, the subject is a livestock animal.

In certain embodiments, the composition is directly applied to the skin, the subject's apparel, and/or clothing.

Another aspect of this invention includes a consumer care product prepared according to the process comprising: (a) mixing a first active ingredient selected from the group consisting of dimethyl anthranilate, methyl anthranilate, ethyl anthranilate, phenylethyl anthranilate and menthyl anthranilate, and fatty acid, (c) optionally at least one additional active ingredient; and (b) adding the mixture to a pharmaceutically suitable topical carrier; wherein the ratio of first active ingredient to fatty acid is about 6:1; 5:1; 4:1 or 3:1. In the preferred embodiment, the ratio of first active ingredient to fatty acid is about 5:1 or 4:1.

In certain embodiments, in the described consumer care product, the fatty acid is selected from the group consisting of oleic acid, ricinoleic acid, linoleic acid palmitic acid, stearic acid, and any mixtures thereof. In other embodiments, the fatty acid is oleic acid.

In certain embodiments, in the described consumer care product, the active ingredient is an anthranilate ester selected from the group consisting of dimethyl anthranilate, methyl anthranilate, ethyl anthranilate, phenylethyl anthranilate and menthyl anthranilate. In an embodiment, the anthranilate ester is methyl anthranilate.

In certain embodiments, the consumer care product, further comprises an additional compound selected from the group consisting of a surfactant, emulsifying agent, a pH modifier, a thickening agent, an anti-oxidant, a preservative, a skin conditioning agent, an emollient, a humectant, a silicone moiety, an anti-inflammatory, a sun blocking agent, a topical anesthetic, vitamins, an antipruritic, an antibiotic, antifungal, an antiseptic, a vasoconstrictor, and any mixtures thereof.

In other embodiments, the emulsifying agent is selected from the group consisting of polysaccharide ethers, polyglycosides, fatty acids, fatty alcohols, amine oxides, water-soluble cellulose, alkyl sulfonates, ethoxylated alkyl phenols, alkanolamides, betaines, zwitterionic surfactants, carboxylated alcohols, carboxylic acids, ethoxylated C12-C15 alcohols, polysorbates, behentrimonium methosulfate, isopropyl myristate and ethoxylated castor oil or any mixtures thereof.

In certain embodiments, the thickening agent is selected from the group consisting of carboxylic acid polymers, crosslinked acrylic acid with allyl ethers of sucrose, crosslinked polyacrylate polymers, polyacrylamide polymers, polysaccharides, gums and mixtures thereof.

In other embodiments, the polysaccharides is selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropylmethylcellulose, methyl hydroxyethylcellulose, microcrystalline cellulose, sodium cellulose sulfate, chitosan and mixtures thereof.

In certain embodiments, in the consumer care product, gums is selected from the group consisting of calcium carrageenan, carnitine, carrageenan, dextrin, gelatin, gellan gum, guar gum, guar hydroxypropyltrimonium chloride, hectorite, hydroxypropyl guar, karaya gum, kelp, locust bean gum, natto gum, potassium carrageenan, sclerotium gum, sodium carrageenan, tragacanth gum, xanthan gum and any mixtures thereof.

In certain embodiments, the pharmaceutically suitable topical carrier comprises water, glycerin, lecithin a fatty acid or any combinations thereof.

In other embodiments of the consumer care product, the fatty acid is selected from the group consisting of oleic acid, ricinoleci acid, linoleic acid, palmitic acid, steric acid or any mixtures thereof.

In certain embodiments of the consumer care product, the sun blocking agent is selected from the group consisting of PABA, PABA esters, butyl PABA, ethyl PABA, ethyl dihydroxypropyl PABA, benzophenones, avobenzone, cinnamates, salicylates anthranilates, ethyl urocanate, homosalate, dibenzoylmethanes, octocrylene, methylbenzylidene camphor, kaolin, talc, petrolatum and metal oxides or any mixtures thereof.

Other aspects of this invention include a moist towelette product comprising a woven or non-woven fabric or cellulosic substrate and the composition comprising an insect or acarid repellant described herein.

Other aspects of this invention include a method for killing an insect or pest, comprising providing a composition comprising an insect or acarid repellant provided herein; and applying the composition to the insect or pest, whereby the insect or pest is killed, wherein the amount applied is sufficient to kill the insect or pest.

Another aspect of this invention includes a method of repelling an insect or acarid from clothing or bedding, comprising: deploying the composition comprising (a) a first active ingredient selected from the group consisting of dimethyl anthranilate, methyl anthranilate, ethyl anthranilate, phenylethyl anthranilate and menthyl anthranilate; (b) a fatty acid; (c) optionally at least one additional active ingredient; and (d) a carrier, to the location whereby the insect is repelled when the insect comes into contact with the composition or vapors from the composition, wherein: the composition is deployed by; applying topically to an article of clothing or bedding; or laundering the article of clothing or bedding with a detergent or fabric softener or both that comprises the composition, or drying the article of clothing or bedding with a fabric softener that comprises the composition.

Another aspect of the present invention is directed to a method of repelling an insect or acarid, comprising deploying an insect repellent composition comprising (a) a first active ingredient selected from the group consisting of dimethyl anthranilate, methyl anthranilate, ethyl anthranilate, phenylethyl anthranilate and menthyl anthranilate; (b) a fatty acid; (c) optionally at least one additional active ingredient; and (d) a carrier, whereby the insect or acarid is repelled when it comes into contact with the composition or vapors from the composition.

In further embodiments of this method, the ratio of a first active ingredient to a fatty acid is 5:1 or 4:1.

In further embodiments of this method, the composition is deployed by: applying the composition topically to an article of clothing of a human; or applying the composition topically to skin or hair of a human; or applying the composition topically to skin or fur of an animal; or laundering an article of clothing of a human with a detergent or fabric softener or both that comprises the composition; or drying an article of clothing of a human with a fabric softener that comprises the composition; or applying the composition to bedding, bed boards, bed slats, a mattress, box springs, furniture, carpeting, baseboards or flooring or a combination thereof; or spraying the composition on to the surface of bedding, bed boards, bed slats, a mattress, box springs, furniture or carpeting; or injecting the composition into the mattress, box springs, furniture or carpeting or a combination thereof; or deploying an absorbent substrate or gel containing the composition in the vicinity of bed boards, bed slats, a mattress, box springs, furniture or carpeting so that vapors from the composition come into contact with a surface of the bed boards, bed slats, a mattress, box springs, furniture or carpeting; or injecting the composition into a wall space.

Another aspect of this invention is the composition comprising an insect or acarid repellant described herein wherein the composition is formulated as a personal care or cosmetic composition.

In certain embodiments, the personal care or cosmetic composition is formulated as a product selected from among insect repellents, skin care products, hair care products, and cleansing products.

In other embodiments, the composition comprising an insect or acarid repellant provided herein is formulated as a household composition.

In certain embodiments, the household composition is formulated as a product selected from among air deodorant/freshener compositions in liquid, gel or solid form, all purpose cleaner compositions, all purpose disinfectant compositions, deodorizing sprays and powders, dish detergents, fabric sizing compositions, fabric softening compositions, fabric static control compositions, hard surface cleanser compositions, hard surface detergents, hard surface sanitizing compositions, linen and bedding spray compositions, pesticide compositions, polishing compositions, laundry detergents, rug and upholstery shampoo compositions, cleaners and deodorizers, tile, toilet and tub cleaning and disinfectant compositions, waxes and cleaning compositions for treating wood floors or furniture, and waxes and cleaning compositions for automobiles.

Another aspect of this invention includes the composition comprising an insect or acaris repellant described herein that is a fabric softening composition selected from among a liquid fabric softener, a fabric softening rinse, a fabric softening sheet, and a fabric softening gel.

Another aspect of this invention includes a fabric treatment sheet comprising the composition comprising an insect or acarid repellant provided herein.

Another aspect of this invention includes a method of treating a structure infested with insects or aracids, comprising: deploying a composition comprising (a) a first active ingredient selected from the group consisting of dimethyl anthranilate, methyl anthranilate, ethyl anthranilate, phenylethyl anthranilate and menthyl anthranilate; (b) a fatty acid; (c) optionally at least one additional active ingredient; and (c) a carrier. In a preferred embodiment, the ratio of the first active ingredient to a fatty acid is about 5:1 or 4:1.

### DETAILED DESCRIPTION OF THE INVENTION

In one aspect, the present invention is directed to a composition comprising an insect and/or acarid repellant amount of a composition comprising: (a) an anthranilate ester; and (b) a fatty acid. In this application, the terms “composition” and “formulation” are interchangeable.

In another aspect, the present invention is directed to a composition comprising an insect or acarid repellant comprising: (a) a first active ingredient selected from the group consisting of dimethyl anthranilate, methyl anthranilate, ethyl anthranilate, phenylethyl anthranilate and menthyl anthranilate; (b) a fatty acid; (c) optionally at least one additional active ingredient and (c) a pharmaceutically suitable topical carrier; wherein the ratio of first active ingredient to fatty acid is about 6:1; 5:1; 4:1; or 3:1.

The anthranilate esters which may be employed include those compounds described in U.S. Pat. No. 2,967,128, which patent is hereby incorporated by reference, and include dimethyl anthranilate, methyl anthranilate, ethyl anthranilate, phenylethyl anthranilate and menthyl anthranilate. Preferred anthranilate esters are dimethyl anthranilate and methyl anthranilate, with methyl anthranilate being particularly preferred.

The fatty acids which may be employed as component (b) include saturated and unsaturated fatty acids containing from 8 to 24 carbon atoms, with fatty acids containing from 13 to 21 carbon atoms being preferred. Illustrative of the fatty acids which may be employed are oleic acid, ricinoleic acid, linoleic acid palmitic acid and stearic acid; with oleic acid being particularly preferred.

Typically, weight ratio of anthranilate ester to fatty acid employed in the compositions of the present invention ranges from 20:1 to 1:10. In certain embodiments, such ratio will range from 5:1 to 1:10. In other embodiments, such ratio is from 5:1 to 1:5. In still other embodiments, the weight ratio of anthranilate ester to fatty acid to from 5:1 or 4:1.

The compositions of this invention may further comprise additional additives conventionally employed in agricultural applications, provided that such additives in the composition are not present in a form that will cause the fatty acid to convert into a salt or other form of surfactant. In some embodiments, this may be accomplished by avoiding the addition of amines such as monoethanolamine, diethanolamine and triethanolamine.

Illustrative of further components which may be included in the compositions of this invention are antioxidant agents which serve to substantially prolong the desirable action of the fatty acid. Such antioxidant agent(s) protect the chemical and physical integrity of the fatty acid against reaction with oxygen and air pollution alone or in the presence of light. There exists an abundance of suitable antioxidants consisting of commercial and specialty chemicals and their combinations, mixtures and proprietary compositions that are well known to those educated in the art.

In certain embodiments, one particular antioxidant agent is selected from a group that includes, but is not limited to, ascorbic acid palmitate, ascorbyl palmitate, butylated p-cresol, tert-butylhydroquinone, butylated hydroquinone monomethyl ether, butylhydroxy-anisole, butylhydroxytoluene, propyl gallate and a tocopherol and combinations thereof. The amount of the antioxidant is preferably from 0.001-0.1% by weight of the total composition.

While the composition can be used neat, i.e. undiluted, it is preferably employed in a diluted form. For example, the composition can be dissolved in a suitable solvent, such as a C₁-C₄ alcohol (for example, methanol, ethanol, isopropyl alcohol, butanol), a ketone such as acetone, an ester such as ethyl acetate or isopropyl myristate, a refined petroleum distillate solution (for example Sunspray® 6E brand from Sunoco Inc.) or other non-reactive solvent that will evaporate, preferably in a short period of time, leaving the active mixture of fatty acid and anthranilate ester. The composition can be formulated as an Emulsifiable Concentrate (EC) with adjuvants, surfactants, stabilizers and preservatives, to be diluted with water for spray application or for application to surfaces such as mopping onto floors, wiping countertops and the like. The repellant compositions can also be formulated as aerosols for application from pressurized containers. The repellant composition can be applied by any typical method, including but not limited to spraying, painting, or dipping the object to be covered in the composition.

Another aspect of this invention is directed to a composition comprising an insect or acrid repellant provided herein, further comprising at least one additional active ingredient selected from the group consisting of pharmaceutically active agents, insecticides, pesticides, fungicides, herbicides, fertilizers, repellant and combinations thereof. Suitable additional active ingredients for the insect or acrid repellant provided herein include the following:

Insecticides: A1) the class of carbamates consisting of aldicarb, alanycarb, benfuracarb, carbaryl, carbofuran, carbosulfan, methiocarb, methomyl, oxamyl, pirimicarb, propoxur and thiodicarb: A2) the class of organophosphates consisting of acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles consisting of ethiprole, fipronil, pyrafluprole and pyriprole; A5) the class of neonicotinoids consisting of acetamiprid, chlothianidin, dinotefuran, imidacloprid, nitenpyram, thiacloprid and thiamethoxam; A6) the class of spinosyns such as spinosad and spinetoram; A7) chloride channel activators from the class of mectins consisting of abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class consisting of bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz, A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides consisting of flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chloranthraniliprole and cyanthraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids consisting of acrinathrin, allethrin, bifenthrin, cyfluthrin, lambda-cyhalothrin, cypermethrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B1) azoles selected from the group consisting of bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chlorophenyl)-2-([1,2,4]triazol-1-yl)-cycloheptanol and imazalilsulfphate; B2) strobilurins selected from the group consisting of azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxv)-5-fluoro-pvrimidin-4-yloxy)-phenyl)-2-methoxyimino-N-methyl-acetamide and 3-methoxy-2-(2-(N-(4-methoxy-phenyl)cyclopropanecarboximidoylsulfanylmethyl)-phenyl)-acrylic acid methyl ester; B3) carboxamides selected from the group consisting of carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamiide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chlorophenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl-methylthiazole-.-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3,4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl)cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-ylcarboxamide, N-3,4′,5′trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methyl pyrazol-4-ylcarboxamide, N-(3′,4′,5′-trifluorobiphenyl-2-yl)-3-(chlorofluoromethyl)-1-methylpyrazol-4-ylcarboxamide,N-(3′,4′,5′-trifluorobiphenyl-2-yl)-3-difluoromethyl-1-methylpyrazol-4-ylcarboxamide, N-(3′,4′,5-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′, 5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4-dichloro-4-fluorobiphenyl-1-yl)-1-methyl-3-trifluoromethyl-1H-pyrazole-4-carboxamide, N-(3′,4′-difluoro-4-fluorobiphenyl-2-yl)-1-methy 1-S-trifluoromethyl-1H-pyrazole-4-carboxamide, N-(3′,4-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4-dichloro-5-fluorobiphenyl-2-yl)-1-methyl-S-difluoromethyl-1H-pyrazole-4-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethyhthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds selected from the group consisting of fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphate, oxolinic acid and piperalin; B5) carbamates selected from the group consisting of mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butylamino)propanoate; or B6) other fungicides selected from the group consisting of guanidine, dodine, dodine free base, iminoctadine, guazatine, antibiotics: kasugamycin, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate-methyl, tolylfluanid, others: cyflufenamid, cymoxanil, dimethirimol, ethirimol, furalaxyl, metrafenone and spiroxamine, guazatine-acetate, iminoctadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine,mildiomycin, oxincopper, prohexadione calcium, N-(cyclopropylmethoxvimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, cloproxydim, cycloxydim, sethoxydimn tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2) acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium, KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chlorthiamid, diphenamid, etobenzanid, fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone, diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC, esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, propyzamid, propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751, JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C16) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts.

Repellants include, but are not limited to, dimethyl anthranilate, methyl anthranilate, ethyl anthranilate, phenylethyl anthranilate and menthyl anthranilate and their environmentally compatible salts, N, N-diethyl-meta-toluamide (DEET), picaridin, oil of lemon eucalyptus, citronella, 2-undecanone, ethyl butyl acetyl aminoproprionate (IR3535). A13-37220, tansy essential oil, amitraz, permethrin, dimethyl phthalate, R-11, and ethyl hexane diol.

In another aspect of the present invention, consumer care products are described for repelling insects and/or acarid pests. Consumer care products may include, but are not limited to, topical compositions for application to the skin, clothing or other material to repel insect and/or acarid pests containing an insect or cararid repellant component such as a pheromone, an essence, an anthranilate ester, O-aminoacetphones, methylbenzamides such as N,N di-ethyl-3-methylbenzamide or N,N diethyl-m-toluamide (DEET), repellant oils such as tee tree oil, lemongrass oil, lemongrass oil, citronella oil, eucalyptus oil, glavender oil, geranium oil, clove oil, cajeput oil and lemon balm oil, and a fatty acid or any mixtures thereof. In certain embodiments of the invention, the product is a composition in liquid or semi solid form and may be aqueous, non-aqueous based or combination thereof and may be in the form of a lotion, cream, ointment, solution, foam, gel, solid stick, aerosol, bar, liquid, paste, powder, roll-on, sheet, spray, stick, tablet, suspension, emulsion, microemulsions, emusifiable concentrates, or the like. The formulation may be designed to be slowly released from a patch or be sprayed from a canister, a delivery apparatus or any suitable delivery system.

The carrier can include all carriers, topically cosmetic carriers, and pharmaceutically acceptable carriers described herein. The carrier can include water, an alcohol, an aldehyde, an alkane, an alkene, an amide, an amine, a diglyceride, an ester, an ether, a glycol ether, a fat, a fatty acid, a glycol ester, a ketone, lanolin, mineral oil, a monoglyceride, paraffin oil, a polyethylene glycol, petrolatum, a propylene carbonate, silicone, tall oils, a terpene hydrocarbon, a terpene alcohol, a triglyceride, finely divided organic solid material, finely divided inorganic solid materials and mixtures thereof. In one embodiment, the carrier can be an alcohol that is selected from among an aromatic alcohol, a C1-C6 monohydric alcohol, C2-C6 polyhydric alcohol, a polyvalent alcohol, and mixtures thereof. In one example, the carrier is an alcohol that is selected from among methanol, ethanol, propanol, butanol, sec-butanol, tert-butanol, and mixtures thereof. In a particular example, the carrier in the provided compositions is ethanol. In another particular example, the compositions contain a carrier that is isopropanol.

In a preferred embodiment, the composition comprising an insect or acarid repellant contains a topical cosmetic carrier and/or pharmaceutically acceptable carrier to form a topical cosmetic or pharmaceutical composition which can be applied to skin.

In certain embodiments, the carrier is an oil selected from the group including, but not limited to, an almond oil, avocado oil, canola oil, cashew oil, cherry seed oil, cocoa butter, coconut oil, corn oil, cottonseed oil, flaxseed oil, grape seed oil, jojoba oil, macadamia nut oil, olive oil, palm oil, palm fruit oil, peanut oil, rapeseed oil, rice bran oil, safflower oil, sesame oil, soybean oil, sunflower oil, and walnut oil and combinations thereof.

In certain embodiments, the carrier can be a diethyl ether, isopropyl ether, n-propyl ether, or a combination thereof. In one example, the carrier is acetone, a methyl ketone, a methyl benzyl ketone, a methyl ethyl ketone, a methyl isopropyl ketone, a methyl butyl ketone, an ethyl ketone, benzyl methyl ketone, and combinations thereof. In other embodiments, the carrier can be ethylene glycol, ethylene glycol monomethyl ether, ethylene glycol dimethyl ether, methylene glycol, methylene glycol monomethyl ether, methylene glycol dimethyl ether, propylene glycol, propylene glycol monomethyl ether, propylene glycol dimethyl ether, butylene glycol, butylene glycol monomethyl ether, butylene glycol dimethyl ether and combinations thereof. In another example, the carrier can be ethylene glycol, ethylene glycol monomethyl ether, ethylene glycol dimethyl ether, methylene glycol, methylene glycol monomethyl ether, methylene glycol dimethyl ether, propylene glycol, propylene glycol monomethyl ether, propylene glycol dimethyl ether, butylene glycol, butylene glycol monomethyl ether, butylene glycol dimethyl ether and combinations thereof.

In certain embodiments, the carrier can be a finely divided organic solid material or finely divided inorganic solid material. In certain preferred embodiments, the carrier can be a dust, a granule, a powder or a salt crystal. In other embodiments, the carrier can be an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, vermiculite, and combinations thereof.

In certain embodiments, the carrier can be an aerosol propellant that is selected from among argon, butane, carbon dioxide, a chlorofluorocarbon, dimethyl ether, a hydrocarbon, a hydrofluorocarbon, isobutane, nitrogen, propane, and a mixture thereof. In a particular example, the aerosol propellant contains difluoromethane, trifluoromethane, difluoroethane, trifluoroethane, tetrafluoroethane or octafluorocyclobutane or a combination thereof.

In other embodiments, the carrier can be a silicone oil that is selected from among cyclical silicones, linear or branched open chained silicones, and combinations thereof. In one embodiment, the silicone oil can be selected from among volatile silicones and non-volatile silicones. In another embodiment, the silicone oil is a volatile silicone oil that is selected from among cyclic polydimethylsiloxanes containing an average of from about 3 to about 9 silicon atoms and linear polydimethylsiloxanes containing an average of from about 3 to about 9 silicon atoms. In another embodiment, the silicone oil is a non-volatile silicone oil that is selected from among dimethicone copolyol, cyclomethicone, polydimethylsiloxane, cyclic dimethyl polysiloxane, aminosilicones, phenylsilicones, diphenyldimethicones, phenyltrimethicones, cyclopentasiloxane, a polymer of dimethyl-siloxane with polyoxyethylene and/or polyoxypropylene, dimethicone copolyol, cetyldimethicone copolyol, cetyl dimethicone, cetyl dimethicone copolyol and dimethiconol and combinations thereof. In further examples, the carrier is a silicone oil that is selected from among polydimethylsiloxane, phenylated silicones, decamethylcyclopentasiloxane, dodecamethylcyclohexasiloxane and octamethylcyclo-tetrasiloxane.

In certain embodiments, the carrier is selected from, but not limited to, a monoglyceride, a diglyceride, an acetylated monoglyceride, or a triglyceride or a combination thereof. In one example, the carrier contains 1-propanol, 2-propanol, 1-butanol, 2-butanol, 2-methyl-1-propanol, 1-pentanol, 2-pentanol, 3-methyl-1-butanol, 3-methyl-2-butanol, ethylene glycol, propylene glycol, 1,4-butanediol, 1,3-butanediol, 2-methyl-1,3-propanediol, 1,4-cyclohexanedimethanol, diethylene glycol, triethylene glycol, PEG-200, PEG-300, PEG-400, PEG-600, 2-methoxyethanol, 2-ethoxyethanol, 2-propoxyethanol, 2-isopropoxyethanol, 2-butoxyethanol, 1-methoxy-2-propanol, 1-ethoxy-2-propanol, 3-methoxy-1-butanol, diethylene glycol monomethyl ether, diethylene glycol monoethyl ether, diethylene glycol mono-n-propyl ether, diethylene glycol mono-isopropyl ether, diethylene glycol monobutyl ether, triethylene glycol monomethyl ether, glycerol, 3-methoxy-1,2-propanediol, or 3-ethoxy-1,2-propanediol.

In another embodiment, the carrier contains bomeol, citronellol or geraniol or a combination thereof. In other embodiments, the carrier contains or is a cyclodextrin that is selected from among an α-cyclodextrin, β-cyclodextrin or γ-cyclodextrin or a combinations thereof.

In certain embodiments, the consumer care product described herein includes a composition comprising an insect or acarid repellant described herein and a delivery vehicle to deliver the composition to a selected location. The delivery vehicle can contain water, an aerosol, a cream, a gel, a lotion, an oil, a spray, a soap, a detergent, a particulate or a substrate. In other embodiments, the delivery vehicle can be a substrate that is a paper, a cloth or a woven or nonwoven material. In one embodiment, the substrate is a nonwoven material that is a flexible sheet containing fibers that are adhesively or thermally bonded. The fibers can be selected from among cellulose ester, cotton, hemp, jute, linen, ramie, rayon, polyamides, polyesters polyolefins, polypropylene, polyvinyl derivatives, silk, sisal and wool and combinations thereof.

In certain embodiments, the delivery vehicle can be a gel that contains a gelling agent that is selected from among agar, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, K-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof. In other embodiments, the delivery vehicle can be a fluid that when dispensed forms a gel in situ. The fluid can contain a gelling agent that selected from among agar, an alginate, a carbomer, carboxyvinyl polymers, dibenzylidene alditols, carboxypolymethylene, collagen, dextrin fatty acid esters, gelatin, hydrogenated styrene/isoprene copolymers, 12-hydroxystearic acid, K-carrageenan, gellan gum, a lower hydroxy cellulose, pectin, polyacrylic acids, styrene-ethylene/propylene block copolymers, styrene-ethylene/butylene-styrene block copolymers, sucrose fatty acid esters and a wax and combinations thereof.

In certain embodiments, the delivery vehicle can be or contain a particulate that is selected from among an alumina, amorphous silica, attapulgite, calcium carbonate, calcium phosphate, a clay, chalk, diatomaceous earths, fumed silica, a kaolin, kieselguhr, magnesium carbonate, microparticulate cellulose, montmorillonite, pyrophyllite, silicic acid, sodium bicarbonate, sodium carbonate, sodium phosphate, sodium pyrophosphate, talc, and vermiculite, and combinations thereof.

In certain embodiments, the insect or acarid repelled by a composition provided herein can be an insect selected from among Siphonaptera insects, such as cat flea (Ctenocephalides felis), dog flea (Ctenocephalides canis), oriental rat flea (Xenopsylla cheopis), human flea (Pulex irritans), chigoe (Tunga penetrans) and European rat flea (Nosopsyllus fasciatus); Anoplura insects, such as Head louse (Pediculus humanus capitis), crab louse (Pthirus pubis), short-nosed cattle louse (Haematopinus eurystemus), sheep louse (Dalmalinia ovis), hog louse (Haematopinus suis), long-nosed cattle louse (Linognathus vituli), cattle biting louse (Bovicola bovis), poultry shaft louse (Menopon gallinae), poultry body louse (Menacanthus stramineus), little blue cattle louse (Solenopotes capillatus), Haematopinus spp., Linognathus spp., Pediculus spp., Phtirus spp, and Solenopotes spp.; Acarina insects, such as bush tick (Haemaphysalis longicomis), Haemaphysalis flava, Dermacentor taiwanicus, American dog tick (Dermacentor variabilis), Ixodes ovatus, Ixodes persulcatus, black legged tick (Ixodes scapularis), lone star tick (Amblyomma americanum), Boophilus microplus, Rhipicephalus sanguineus, Ixodes holocyclus, western black legged tick (Ixodes pacificus), Dermacentor andersoni, Ambryomma maculatum, ear mite (Octodectes cynotis), Psoroptes spp., Chorioptes spp., Otodectes spp., Sarcoptes spp., Notoedres spp., Knemidocoptes spp., Sacroptes scabiei, Demodex spp., follicle mite (Demodex canis), northern fowl mite (Omithonyssus sylviarum), poultry red mite (Dermanyssus gallinae), Trombicula spp., Leptotrombidium akamushi, Omithodorus hermsi, Omithodorus turicata, Omithonyssus bacoti. Acarapis spp., Cheyletiella spp., Omithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Cytodites spp, and Laminosioptes spp.; Heteroptera insects, such as common bedbug (Cimex lectularius), tropical bedbug (Cimex hemipterus), Reduvius senilis, Triatoma spp. Rhodnius spp., Panstrongylus spp., and Arilus critatus; and Mallophage (Amblycera and Ischnocera) insects, such as Trimenopon spp., Menopon spp., Trinoton spp., Bovicola spp., Wemeckiella spp., Lepikentron spp., Trichodectes spp, and Felicola spp.

In certain embodiments, the insect or acarid repelled by a composition provided herein can be an insect selected from among ants, bedbugs, carpet beetles, centipedes, chiggers, drain flies, dust mites, biting mites, earwigs, fleas, flies, gnats, hornets, lice, millipedes, mites, mosquitoes, roaches, scabies, silverfish, spiders, stinkbugs, termites, ticks, wasps, weevils and yellow jackets. The compositions comprising insect or acarid repellants provided herein, in certain embodiments, can be formulated for delivery of for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days.

Certain embodiments also include fabric treatment sheets containing a woven or nonwoven sheet or other substrate, including but not limited to cellulosic substrate, that is coated or impregnated with a fabric treatment composition and a composition comprising an insect or acarid repellant provided herein. The fabric treatment composition can be a detergent composition or a fabric softening composition. In some embodiments, the fabric treatment composition is a detergent composition that contains a detergent selected from among anionic surfactants, nonionic surfactants, zwitterionic surfactants, ampholytic surfactants and cationic surfactants and mixtures thereof. In other embodiments, the fabric treatment composition is a fabric softening composition that contains monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries or (C8-C24) fatty acid amides or any combination thereof.

Certain embodiments include liquid fabric treatment compositions containing a fabric softener or fabric conditioner and a composition comprising an insect or acarid repellant provided herein. The fabric softener can be selected from among monomethyl trialkyl quaternaries, imidazolinium quaternaries, dimethyl alkyl benzyl quaternaries, dialkyl dimethyl quaternaries, methyl dialkoxy alkyl quaternaries, diamido amine-based quaternaries and dialkyl methyl benzyl quaternaries and (C8-C24) fatty acid amides and combinations thereof. In some embodiments, the fabric conditioner contains an anti-static agent, a brightening agent, a bodying agent, a soil-release agent, a wrinkle-release agent or a combination thereof. In certain embodiments, the fabric condition is an anti-static agent that contains a tertiary amine, a quaternary amine, aluminum stearate or a combination thereof. In another example, the fabric conditioner is a brightening agent that contains hydrogen peroxide, potassium permanganate, sodium peroxide, sodium perborate, disulfonated diaminostilbene optical brightener compounds and triazole optical brightener compounds. In yet another embodiment, the fabric conditioner is a bodying agent that is selected from among carboxymethyl cellulose, hydroxyethylcellulose, starch, polyvinyl acetate and combinations thereof. In yet another embodiment, the fabric conditioner is a wrinkle release agent that contains polyvinyl acetate.

Certain embodiments include fabric refresher spray compositions containing a composition for killing or repelling pests provided herein wherein the active ingredient is present at a concentration of from at or about 0.1% to at or about 5%, or in an amount of at or about 1% to at or about 10%, or greater than 10%, or greater than 15%, or greater than 20% or greater than 25% by weight of the composition. The fabric refresher spray compositions can further contain a cyclodextrin; and/or an ampholytic surfactant, an anionic surfactant, a cationic surfactant, a nonionic surfactant or a zwitterionic surfactant or a combination thereof, and/or water.

Certain embodiments include moist towelette products containing a substrate and a solution or emulsion of a composition comprising an insect or acarid repellant provided herein wherein the active ingredient is present in an amount of at least 0.1% by weight of the solution. The moist towelette products can further contain a surfactant containing, but not limited to, cocamidopropyl betaine, coco-glucoside or decyl glucoside or combinations thereof. In one embodiment, the substrate in the moist towelette product is a nonwoven fabric or cellulosic material.

Certain embodiments also include packaged pest repelling compositions that contain a container holding a composition comprising an insect or acarid repellant provided herein or an absorbent sheet impregnated with a composition comprising an insect or acarid repellant provided herein. Other embodiments include aerosol propellant pressurized sprayable pest repellents or pesticide products containing a composition comprising an insect or acarid repellant provided herein. The propellant in the aerosol propellant pressurized sprayable pest repellents or pesticide products can contain carbon dioxide, propane, butane or a mixture thereof.

Certain embodiments include methods of repelling an insect or pest from a location using a composition comprising an insect or acarid repellant provided herein. In the provided methods, a composition comprising an insect or acarid repellant provided herein is deployed at the location in an insect or pest repelling amount, wherein the insect or pest is repelled when the insect or pest comes into contact with the composition or vapors from the composition. In certain embodiments, the composition can be deployed by atomizing, brushing on, coating, dipping, drenching, dripping, dusting, foaming, infusing, injecting into or onto, pouring, rolling on, scattering, spraying, spreading, sprinkling or wiping the composition onto at least a portion of the location.

In certain embodiments of the method for repelling an insect or pest from a location, the location is the surface of the body of a human or animal. For example, the animal can be a companion animal or a farm animal. In other embodiments of the method, the composition is deployed by applying topically to an article of clothing of a human. In other embodiments of the method, the composition comprising an insect or acarid repellant provided herein is deployed by laundering an article of clothing of a human with a detergent or fabric softener or both that contains the composition. In yet another embodiment of the method, the composition is deployed by drying an article of clothing of a human with a fabric softener that contains the composition. The fabric softener can be provided as a dryer sheet or gel. In another embodiment of the method, the location is a surface that is skin, hair or fur and the composition is deployed by applying topically to the skin, hair or fur. The topically applied composition can be provided as an aerosol, a solution, an emulsion, an oil, a lotion, a soap, a spray, or a gel. In yet another embodiment of the method, the composition is provided to a surface that is skin, hair or fur in a form selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products, anti-wrinkle products, hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars, shower gels and shampoos.

In certain embodiments of the method for repelling an insect or pest from a location, the location is a bedding location. The composition comprising an insect or acarid repellant provided herein can be deployed onto bedding, bed boards, bed slats, a mattress, box springs, furniture, carpeting, baseboards or flooring or a combination thereof by atomizing, coating, dipping, drenching, dripping, dusting, foaming, infusing, injecting into or onto, pouring, rolling on, scattering, spraying, spreading, sprinkling or wiping. In another embodiment of the method, the composition comprising an insect or acarid repellant provided herein is deployed by spraying the composition on to the surface of bedding, bed boards, bed slats, a mattress, box springs, furniture, carpeting, baseboards or flooring or a combination thereof. For example, the composition comprising an insect or acarid repellant provided herein can be deployed by injecting the composition into the mattress, box springs, furniture, carpeting, baseboards or flooring or a combination thereof. In certain embodiments of the method, the location contains a wood structure, wooden object or wall space.

In other embodiments of the method, the location is selected from among an air supply duct, an attic, an awning, a basement, a cellar, a crawlspace, a deck, a dock, a garage, a hamper, a heating vent, a home foundation, a linen storage closet, a pool deck, roof tiles, a shipping container, a storage unit, a suitcase, a walkway and a wall space and the composition is deployed by atomizing, coating, dipping, drenching, dripping, dusting, foaming, infusing, injecting into or onto, pouring, rolling on, scattering, spraying, spreading, sprinkling or wiping the composition onto or into at least a portion of the location. For example, the composition comprising an insect or acarid repellant provided herein is applied by spraying the composition onto a surface or is provided in the form of a powder and is applied by sprinkling the powder composition onto a surface. In yet other embodiments of the method for repelling an insect or pest from a location the composition comprising an insect or acarid repellant provided herein is deployed by providing an absorbent substrate or gel containing the composition and positioning it in the location. In one example, the absorbent substrate contains a nonwoven fabric or cellulosic material.

In certain embodiments, the insect or pest can be selected from among, but not limited to, ants, bedbugs, carpet beetles, centipedes, chiggers, drain flies, dust mites, earwigs, fleas, flies, gnats, hornets, lice, millipedes, mites, mosquitoes, roaches, scabies, silverfish, spiders, stinkbugs, termites, ticks, wasps, weevils and yellow jackets. For example, in the provided method, the insects or pests are ants that are selected from among Argentine ants, black ants, carpenter ants, fire ants, odorous house ants, pavement ants and pharaoh ants. In another embodiment, the insects or pests are lice that are selected from among head lice, body lice, pubic lice and nits thereof.

In certain embodiments, a composition comprising an insect or acarid repellant provided herein can be deployed by applying topically to an article of clothing of a human; or applying topically to skin or hair of a human; or applying topically to skin or fur of an animal. In some embodiments, the animal can be selected from among a bovine, canine, caprine, cervine, cricetine, feline, galline, equine, lapine, murine, musteline and ovine. For example, the animal is a companion animal. In other embodiments, the composition comprising an insect or acarid repellant provided herein can be deployed by laundering an article of clothing of a human with a detergent or fabric softener or both that contains the composition; or drying an article of clothing of a human with a fabric softener that contains the composition.

Certain embodiments include methods for repelling bedbugs, where a composition comprising an insect or acarid repellant provided herein is deployed by applying directly to the surface of the bedbugs or to bedding, bed boards, bed slats, a mattress, box springs, furniture, carpeting, baseboards or flooring or a combination thereof. For example, application of the composition comprising an insect or an acarid repellant provided herein can include spraying the composition on to the surface of bedding, bed boards, bed slats, a mattress, box springs, furniture or carpeting; or injecting the composition into the mattress, box springs, furniture or carpeting or a combination thereof; or deploying an absorbent substrate or gel containing the composition in the vicinity of bed boards, bed slats, a mattress, box springs, furniture or carpeting so that vapors from the composition come into contact with a surface of the bed boards, bed slats, a mattress, box springs, furniture or carpeting; or injecting the composition into a wall space. In certain embodiments, the absorbent substrate contains a nonwoven fabric or cellulosic material.

In one embodiment of the provided method for repelling bedbugs, the composition comprising an insect or acarid repellant provided herein contains from at or about 0.01% to at or about 10%, or greater than 10%, or greater than 15%, or greater than 20%, or greater than 25%, or up to 99% of an active ingredient, fatty acid, and a carrier. In some examples, the composition is formulated for delivery of an active ingredient for at least 4 days. In any of the methods provided herein, the composition can be formulated for delivery of an active ingredient for at least 1 day, or at least 2 days, or at least 3 days, or at least 4 days, or at least 5 days, or at least 6 days, at least 7 days, or at least 8 days, or at least 9 days, or at least 10 days, or at least 11 days, or at least 12 days, at least 13 days, for at least 14 days, or at least 15 days, or at least 16 days, or at least 17 days, or at least 18 days, or at least 19 days, at least 20 days, or at least 21 days, or at least 22 days, or at least 23 days, or at least 24 days, or at least 25 days, at least 26 days, or at least 27 days, or at least 28 days, or at least 29 days, or at least 30 days, or at least 31 days, or at least 45 days, or at least 60 days or at least 75 days or at least 90 days.

Certain embodiments include a method of preventing skin injury due to biting insects or pests by providing a composition for killing or repelling insects provided herein and applying the composition to a surface, wherein the insect or pest is repelled from the surface when it comes into contact with the composition comprising an insect or acarid repellant provided herein or with vapors from the composition comprising an insect or acarid repellant provided herein. In some embodiments the surface is clothing or bedding and the composition comprising an insect or acarid repellant provided herein can be applied to the surface by atomizing, coating, dipping, drenching, dripping, dusting, foaming, infusing, injecting into or onto, pouring, rolling on, scattering, spraying, spreading, sprinkling or wiping an amount of the composition onto the surface. In other embodiments of the provided method, the composition comprising an insect or acarid repellant provided herein is applied by washing the clothing or bedding with the composition comprising an insect or an acarid repellant provided herein that is provided as a detergent composition or a fabric softener composition or both. In other embodiments of the provided method, the composition comprising an insect or acarid repellant provided herein can be applied by drying the clothing or bedding with the composition that is provided as a fabric softener composition. In some embodiments of the method, the surface is bed boards, bed slats, a mattress, box springs, furniture or carpeting and the composition can be applied by atomizing, coating, dipping, drenching, dripping, dusting, foaming, infusing, injecting into or onto, pouring, rolling on, scattering, spraying, spreading, sprinkling or wiping the composition onto the surface. In yet another embodiment of the method, the composition comprising an insect or acarid repellant provided herein can be provided in the form of a powder and is applied by sprinkling the powder composition onto the surface.

In some embodiments of a method for preventing skin injury due to biting insects or pests, the surface is skin or hair of a human and the composition comprising an insect or acarid repellant provided herein can be applied topically to the skin or hair. In an embodiment, the composition comprising an insect or acarid repellant provided herein can be provided as an aerosol, a solution, an emulsion, an oil, a lotion, a soap, a spray, or a gel. In yet other embodiment, the composition is provided in a form selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products, anti-wrinkle products, hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays, soaps, foaming bath products, hand/body/facial cleansers, astringent cleansers, anti-acne products, shampoos, body shampoos, synthetic detergent bars, shower gels and shampoos. The insect or pest can be selected from ants, bedbugs, chiggers, fleas, lice, mites, mosquitoes, roaches, scabies, and ticks.

In at least one embodiment, the disclosed invention includes a consumer care product prepared according to the process comprising (a) mixing an active ingredient and fatty acid; and (b) adding the mixture to a pharmaceutically suitable topical carrier.

In at least one embodiment, the disclosed invention includes a repellent pheromone such as necromone or an anthranilate ester, alone or in combination with aromatic oils. Preferably, the repellent pheromone is present in the amounts of 4% to 10% w/w. In another embodiment, the fatty acids of the presently claimed consumer care product include saturated and unsaturated fatty acids containing from 8 to 24 carbon atoms, with fatty acids containing from 13 to 21 carbon atoms being preferred. Illustrative of the fatty acids which may be employed are oleic acid, ricinoleic acid, linoleic acid palmitic acid and stearic acid; with steric and oleic acids being particularly preferred.

In one embodiment, the present invention concerns a topical skin care composition that contains water, a surfactant, emulsifying agent, a skin conditioning agent or an emollient, a humectant, silicon, an anti-inflammatory, a sun blocking agent, a topical anesthetic, vitamins, an antipruritic, an antibiotic, antifungal, an antiseptic, a vasoconstrictor, and other suitable topical or cosmetic excipients such as a pH modifier, a thickener, an anti-oxidant, a preservative and the like.

In certain embodiments, the composition further contains a dispersing agent that is selected from among, but not limited to, a surfactant, polyvinyl pyrrolidone, polyoxyethylated castor oil, a polyoxyethylene sorbitan ester, alkylnaphthalene sulfonate, alkylbenzenesulfonate, polyoxyethylene, polycarboxylate, lignin sulfonate, sodium silicate, potassium silicate, methylcellulose, carboxymethyl cellulose, hydroxypropylcellulose, hydroxypropyl-methylcellulose, gum arabic, a polyacrylate, and an acrylic/maleic copolymers and combinations thereof.

In at least one embodiment, the composition contains a surfactant in amounts of 0 to 40% w/w, or an emulsifying agent including, but not limited to, such compounds as anionics, cationics, non-ionic, amphoteric, zwitteronics, and combinations thereof. Exemplary of such surfactants and/or emulsifiers suitable for use with the present disclosure include, but are not limited to, esters, amides, polysaccharide ethers, polyglycosides, fatty acids, fatty alcohols, amine oxides, water-soluble cellulose derivatives, alkyl sulfonates, ethoxylated alkyl phenols, alkanolamides, betaines, zwitterionic surfactants, carboxylated alcohols, carboxylic acids, ethoxylated alcohols, preferably ethoxylated C₁₂-C₁₅ alcohols (ex. Rhodasurf® L-9 brand); nonionic surfactants such as polysorbate 20, polysorbate 80, alkoxylated alcohol, a dialkoxylated alcohol, an alkoxylated dialkylphenol, an alkylpolyglycoside, an alkoxylated alkylphenol, an alkoxylated glycol, an alkoxylated mercaptan, an alkylamine salt, an alkyl quaternary amine salt, a glyceryl or polyglyceryl ester of a natural fatty acid, an alkoxylated glycol ester, an alkoxylated fatty acid, an alkoxylated alkanolamide, a polyalkoxylated silicone and an N-alkyl pyrrolidone and combinations thereof, anionic surfactants such as DEA phosphate, alkyl sulfonate surfactants, a linear alkylbenzene sulfonic acid, a branched alkylbenzene sulfonic acid a C12 to C18 alkylsulfate, C12-C18 alkyl alkoxy sulfate, C12-C18 alkyl methyl ester sulfonate and combinations thereof, cationic surfactants such as behentrimonium methosulfate, alkylamine, an alkyl diamine, an alkyl polyamine, a mono- or di-quaternary ammonium salt, a monoalkoxylated amine, a dialkoxylated amine, a monoalkoxylated quaternary ammonium salt, a dialkoxylated quaternary ammonium salt, an etheramine, an amine oxide, an alkoxylated amine oxide and a fatty imidazoline and the like and derivatives thereof.

In at least one embodiment, the formulation is in the form of a personal care composition, such as a foaming composition that contains single emulsifier or a combination of two or more emulsifiers. In at least one embodiment, the formulation contains both an aqueous and an organic phase, wherein the organic phase contains a surfactant and an organic diluent. In one embodiment, the organic phase may comprise the active ingredient, isopropyl myristate and ethoxylated castor oil or the like (ex. Agnique®BP-4-3101 brand).

The dispersing agent can be present in the provided compositions in an amount of at or about 0.002% to at or about 50% by weight of the composition. In some examples, the dispersing agent is present in an amount of at or about 0.025% to at or about 25% by weight of the composition. In other examples, the dispersing agent is present in an amount of at or about 0.01% to at or about 15% by weight of the composition. In yet other examples, the dispersing agent is present in an amount of at or about 0.05% to at or about 10% by weight of the composition, for example, in an amount at or about at least 0.05%, 0.06%, 0.07%, 008%, 0.09%, 0.1%, 0.15%, 0.2%, 0.25%, 0.3%, 0.35%, 0.3%, 0.35%6, 0.5%, 0.55%, 0.6%, 0.65%, 0.7%, 0.75%, 0.8%, 0.85%, 0.9%, 0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%, 2.25%, 2.3%, 2.35%, 2.4%, 2.45%, 2.5%, 2.55%, 2.6%6, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%6, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%, 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%, 7%, 7.05%, 7.1%, 7.15%, 7.2%, 7.25%6, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%6, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95% and 10% by weight of the composition.

In another embodiment, the composition may contain one or more thickening agents. In one embodiment, a thickening agent is present at a level of from about 0 to about 40%, preferably from about 0.1% to about 30%, and more preferably from about 0.25% to about 15%, by weight of the composition. Non-limiting classes of thickening agents include those selected from the following: carboxylic acid polymers useful such as carbomers, crosslinked acrylic acid with allyl ethers of sucrose or pentaerythritol, generally available as the Carbopol series from B.F. Goodrich. Other thickening agent include crosslinked polyacrylate polymers, polyacrylamide polymers, polysaccharides or gelling agents such as cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Other thickening agents include gums such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum (Kelzan®), and mixtures thereof.

In at least one embodiment the topical composition may further contain one or more topical anesthetics (or a plurality of topical anesthetics). Preferably, the topical anesthetic is lidocaine. In other embodiments, in lieu of, or in addition to, lidocaine, one or more of the following may be used; benzocaine, butamben, dibucaine, oxybuprocaine, pramoxine, proparacaine, proxymetacaine, tetracaine, amethocaine, propoxycaine, mepivacaine, bupivacaine, and the like, and mixtures thereof and equivalents.

In at least another embodiment, the anti-inflammatory agent can be a nonsteroidal such as diclofenac, salicylate naproxen, ketorolac and the like or a corticosteroid a such as hydrocortisone, prednisone, cortisol; dexamethasone, alcometasone; hydrocortisone 21-acetate; hydrocortisone 17-valerate; hydrocortisone 17-butyrate; bethamethasone 17,21-dipropionate; descinolone acetonide; bethamethasone valerate; prednisolone; fluorometholone; flucloronide; cortisone acetate; flunisolide acetate; triamcinolone acetonide; flucinonide; desconide; and the like; and mixtures thereof; and equivalents thereof. It is to be understood that any anti-inflammatory agent and/or topical corticosteroid may be used in the composition of the present invention. In other embodiments, the anti-inflammatory agent/topical corticosteroid is omitted.

In yet another embodiment, the composition comprises one or more anti-fungal agents. Preferably, the anti-fungal agent is nystatin; lotrimazole; fluconazole; ketoconazole; itraconazole; lotrimin; canesten; mycosporin; mycelex; clotrimazol; mykosporin; empecid; chlotrimazole, and the like, and mixtures thereof and equivalents thereof.

In yet another embodiment, the composition may further comprise a sun block or UV light blocker such as para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), avobenzone, cinnamates (and octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), trolamine salicylate, anthranilates, ethyl urocanate, homosalate, dibenzoylmethane derivatives (e.g., avobenzone), octocrylene, methylbenzylidene camphor, etc, and the brands Tinosorb M™, Tinosorb S™, Tinosorb A2B™, Neo Helipan™, Mexoryl XL™, Uvinul T™, Parsol SLX™. Non-limiting examples of physical sunblocks include, kaolin, talc, petrolatum and metal oxides (e.g., titanium dioxide and zinc oxide or the like. Additional examples of skin conditioning agents, UV light blockers, thickeners, antioxidants, and pharmaceutically and cosmetically acceptable ingredients may be found in such texts as McCutcheons Emulsifiers and Detergents as well as (Vol 2) Functional Materials, both the North American Editions and the International Editions, published yearly by McCutcheon Publications, both of which are hereby incorporated by reference in their entireties.

In yet another embodiment, the topical composition can further contain an skin conditioning agent or an emollient including a plant and/or a herbal component to make the external layers of the skin softer and more pliable. The emollient may be aloe vera extract, rose oil (i.e., the essential oils extracted from various types of rose), arnica extract, chamomile extract; chamomile flower extract; cetearyl alcohol; isopropyl myristate; triglyceride; myristic acid; palmitic acid; PEG-60 hydrogenated castor oil; glceryl linoleate; cyclomethicone; dimethicone; decyl oleate; stearic acid; lanolin; plant oils; shea butter; cocoa butter; algae extract; avocado oil; seed oil; buckthom; calendula; camellia oil; capsaicin; castor oil; chamomile oil; lemongrass extract; lime oil; marula oil; olive oil; panthenol; corn oil; flaxseed oil; ginseng extract; haxel oil; pomegranate seed oil; rice bran oil; ginger; and the like, and mixtures thereof. In another embodiment, the topical composition may contain a vitamin such as vitamin A, B, C, E, beta-carotene; vitamin B complexes; vitamin B12, an antioxidant, a mineral such as sodium, potassium, calcium or the like. In other embodiments, the vitamin or minerals may be omitted.

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl) phosphite

Examples of preservatives include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal, an azole, benzisothiazolin-3-one, benzalkonium quaternary compounds, benzyl alcohol, borates, 2-bromo-2-nitro-propane-1,3-diol, butylparaben, 5-chloro-2-methyl-4-isothiazolin-3-one, chlorphenesin, chlor-oxylenol, diazolidinyl urea, a dimethyl-benzylalkyl-ammonium chloride, ethyl paraben, formaldehyde, glutaraldehyde, halogenated salicylanilides, hexachlorophene, isobutyl-paraben, isothiazolin-3-one, 2-methyl-4-isothiazoline-3-one, methylparaben, mono-chloracetamide, neomycin sulfate, o-phenylphenol and salts thereof, phenoxyethanol, propionic acid and salts thereof, propylparaben, sodium benzoate, sorbic acid and salts thereof, tebuconazole and triazoles, or combinations thereof.

In certain embodiments, the compositions provided herein can further contain a colorant that is selected from among, but not limited to, a dye or pigment. In one example, the colorant is present in an amount at or about 0.0001% to at or about 1% by weight of the composition. In another example, the colorant is present in an amount at or about 0.0005% to at or about 0.5% by weight of the composition.

The compositions for killing or repelling pests provided herein can be formulated as a personal care or cosmetic composition. In some applications, the personal care or cosmetic composition is formulated as a product selected from among insect repellents, skin care products, hair care products, and cleansing products. In some examples, the personal care or cosmetic composition is a skin care product that is selected from among skin conditioners, hand/body/facial lotions, skin moisturizers, skin toners, skin sanitizers, skin cleansing compositions, skin soothing and lubricating compositions, sunscreen products, anti-aging products, tanning products, self-tanning products, after-sun products, masking products and anti-wrinkle products. In other examples, the personal care or cosmetic composition is a hair care product that is selected from among hair conditioners, hair styling gels, hair anti-dandruff compositions, hair growth promoter compositions, hair lotions, hair tonics, rinses, conditioners, hair colorant compositions, hair anti-frizzing agent compositions, hair shining compositions, mousses, styling gels, hair pomade products and hair sprays. In other examples, the personal care or cosmetic composition is a cleansing product that is selected from among soaps, foaming bath products, hand/body/facial cleansers, astringent cleansers, anti-acne products, shampoos, body shampoos, synthetic detergent bars, shower gels and shampoos.

The compositions for killing or repelling pests provided herein can be formulated as a household care composition. In some examples, the household care composition is formulated as a product that is selected from among air deodorant/freshener compositions in liquid, gel or solid form, all purpose cleaner compositions, all purpose disinfectant compositions, deodorizing sprays and powders, dish detergents, fabric sizing compositions, fabric softening compositions, fabric static control compositions, hard surface cleanser compositions, hard surface detergents, hard surface sanitizing compositions, linen and bedding spray compositions, pesticide compositions, polishing compositions, laundry detergents, rug and upholstery shampoo compositions, cleaners and deodorizers, tile, toilet and tub cleaning and disinfectant compositions, waxes and cleaning compositions for treating wood floors or furniture, and waxes and cleaning compositions for automobiles. In one example, the household care composition is a fabric softening composition that is selected from among a liquid fabric softener, a fabric softening rinse, a fabric softening sheet, and a fabric softening gel.

In at least one embodiment, the topical composition can contain a repellent component from 0.01% to about 99% by weight. In at least one embodiment, the repellent component can range from 0.01 to 25 wt %. In one preferred embodiment, the repellant is methyl anthranilate (also may be referred herein as MA) in ranges of about 2 to 15 wt %. In a more preferred embodiment, the amount of methyl anthranilate is below 8% and can be applied to a subject's skin in an amount up to about 5000 mg/kg.

In at least one embodiment, the topical formulation contains an organic and an aqueous phase. In one embodiment, the organic phase contains the repellant and optionally a surfactant or an emulsifying agent in combination with a fatty acid. In a preferred embodiment, the aqueous phase contains water, glycerin, lecithin, a C₁-C₆ alcohol or any combinations thereof. In an alternative embodiment, the aqueous phase may contain a thickening agent such as xanthan gum. In one embodiment, the aqueous phase may contain water in an amount ranging from about 5% to about 50%, preferably in the ranges of 40% to 45% and xanthan gum in an amount of 0% to about 40%, preferably in the range of 20% to 30%, all % in % w/w.

In at least one embodiment, the topical formulation is in the form of an oil in water emulsion containing MA, oleic acid, steric acid, ethoxylated alcohol (e.x. Rhodasur® L-9 brand), glycerin and water. In at least one preferred embodiment, the MA is in the ranges of about 2% to about 8%, preferably 3.35% to 6.7%. In another embodiment, oleic acid is in the ranges of about 0.5 to 3%, preferably 0.065% to 1.3%. In another embodiment, steric acid can be in the ranges of about 1 to 10 wt %, preferably in the ranges of 1.5 to 4.0 t %, all % are % w/w.

In at least another embodiment, the topical formulation contains diethylhexyl adipate, homosalate, octocrylene, octisalate, avobenzone, bemotrizinol, cyclopentasiloxane, cyclohexasiloxane, necromone and ethanol. In yet another embodiment, the formulation is in the form of a lotion containing water and lecithin in the aqueous phase, while containing oleic acid, methyl anthranilate and Caproyl 90™ in the organic phase. In yet another embodiment, the topical formulation is in the form of a body spray containing C₁₂-C₁₅ alkyl benzoate, triethylhexyl citrate, isododecane, necromone, polyamide-3 and denatured alcohol.

In at least one embodiment, the consumer care product comprises MA in an amount ranging from about 1% to about 10%; about 3% to about 7.5%; 3.35% to about 6.7%. In another embodiment, oleic acid (also referred herein as “OA”) is in an amount ranging from about 0.1% to about 2.5%, preferably about 0.45% to about 1.3%; steric acid in an amount ranging from about 1% to about 8%, preferably about 2.0% to about 4.0%; an ethoxylated C₁₂-C₁₅ alcohol in an amount ranging from about 1.5% to about 12%; preferably about 3.0% to about 6%, glycerin in amount of about 2.5 to about 15%; preferably about 5% and a solvent such as water in an amount of 25% to 85%, preferably about 75%, all % are % w/w.

Another aspect of the present invention is directed to a method of repelling insect and/or acarid pests employing the anthranilate ester/fatty acid compositions described above. Such compositions provide effective repellent activity against a number of insect orders including Hymenoptera, including pavement ant, carpenter ant, Argentine ant, little fire ant, ghost ant, cold tolerant ant, wood ant, Asian needle ant, odorus ant, rover ant, fire ant, bigheaded ant, honeybee, carpenter bee, bumble bee, small carpenter bee, European paper wasp, German yellowjacket and bald-faced hornet; Blattodea including German roach, Eastern subterranean termite; Hemiptera including bed bugs and brown marmorated stink bugs; Diptera including Aedes and Anopheles mosquitoes; Coleoptera including corn rootworm, sawtoothed beetle and confused flour beetle; Siphonaptera including cat flea and Lepidoptra including tobacco budworm. Repellent activity has also been demonstrated against brown dog ticks.

The compositions of the present invention have been shown not to exhibit repellent activity against a number of bird species including hummingbirds, grackles, sparrows, woodpeckers, jays, chickadees, titmouse, wrens, starlings, cardinals, finches, flickers and doves.

The repellent composition of this invention is suitable for excluding pest presence and infestation even in sensitive areas such as food storage and preparation areas, kitchen floors, cabinets, drawers, dining areas and play areas. The repellant composition can be used in other applications such as application in and around buildings (homes, garages, barns, restaurants, wineries, and industrial buildings), electrical boxes, gardens, agricultural fields, golf courses, trash cans, decks and patios and to repel pests at outdoor social events such as outdoor parties, picnics and the like. The composition can be applied onto or incorporated into materials such as paper, cellulose or natural sponges, disposable wipes, trashbags, cloth, for example, clothing, table cloths, placemats, camping gear (backpacks, tents, tarps or netting) or incorporated into plastics such as trash bags or plastic table coverings. The repellant composition can be sprayed onto bird feeders or onto bird seed to repel competitive pests without repelling birds.

In at least another embodiment, the present invention is directed to a method of repelling an insect from a subject comprising topically administering to a subject in need thereof a composition comprising at least one insect or acarid repellant component containing an anthranilate ester and a pharmaceutically suitable topical carrier. In one embodiment, the subject is a mammal such as human, dogs, other domesticated animals or live cattle.

In another embodiment, the present topical compositions can be directly applied to the skin or a user's apparel and clothing. In yet another embodiment, the insect orders including Hymenoptera, including pavement ant, carpenter ant, Argentine ant, little fire ant, ghost ant, cold tolerant ant, wood ant, Asian needle ant, odorus ant, rover ant, fire ant, bigheaded ant, honeybee, carpenter bee, bumble bee, small carpenter bee, European paper wasp, German yellowjacket and bald-faced hornet; Blattodea including German roach, Eastern subterranean termite; Hemiptera including bed bugs and brown marmorated stink bugs; Diptera including Aedes and Anopheles mosquitoes; Coleoptera including corn rootworm, sawtoothed beetle and confused flour beetle; Siphonaptera including cat flea and Lepidoptra including tobacco budworm and brown dog ticks.

Preferred embodiments of the invention include: on-skin formulations containing a mixture of oleaic acid and methyl anthranilate, in the various proportions recited herein; household products such as sprays containing a mixture of oleaic acid and methyl anthranilate, in the various proportions recited herein; and household products such as clothing treatments containing a mixture of oleaic acid and methyl anthranilate, in the various proportions recited herein.

The modifier “about” is used herein to indicate that certain preferred operating ranges, such as ranges for molar ratios for reactants, material amounts, and temperature, are not fixedly determined. The meaning will often be apparent to one of ordinary skill. For example, a recitation of a concentration of about 200 grams per liter in reference to, for example, a formulation would be interpreted to include other like concentrations that can be expected to provide similar effect for the concentration, such as 180 grams per liter or 220 grams per liter. Where guidance from the experience of those of ordinary skill is lacking, guidance from the context is lacking, and where a more specific rule is not recited below, the “about” range shall be not more than 10% of the absolute value of an end point or 10% of the range recited, whichever is less.

All lists used to describe the invention disclosed herein are open ended and contain other items, unless specifically stated otherwise.

### Formulation Examples

Formulation A: The following formulation was prepared to test insect repellency (% by weight): 30% oleic acid, 10% methyl anthranilate and 60% Sunspray 6E oil (Sunoco Oil Company) were blended until homogenous. In another embodiment, Sunspray 6E oil can be replaced with any reasonable alternative light parafinnic oil.

Formulation B: The following formulation was prepared to test insect repellency (% by weight): 9% oleic acid, 3% methyl anthranilate and 88% Sunspray 6E oil (Sunoco Oil Company) were blended until homogenous.

Formulation C: The following formulation was prepared to test insect repellency (% by weight): 3% oleic acid, 1% methyl anthranilate and 96% Sunspray 6E oil (Sunoco Oil Company) were blended until homogenous.

Formulation D: The following formulation was prepared to test insect repellency (% by weight): 0.9% oleic acid, 0.3% methyl anthranilate and 98.8% Sunspray 6E oil (Sunoco Oil Company) were blended until homogenous.

Formulation E: The following formulation was prepared to test insect repellency (% by weight): 0.3% oleic acid, 0.1% methyl anthranilate and 99.6% Sunspray 6E oil (Sunoco Oil Company) were blended until homogenous.

Formulation F: The following formulation was prepared to test insect repellency (% by weight): 30% oleic acid, 10% methyl anthranilate, 52% methyl laurate, 4% Agnique® ABS 60CB (Calcium Dodecyl Benzene Sulfonate from Cognis Corporation), 2% Agnique® CSD-40 (EO/PO block co-polymer from Cognis Corporation), and 2% Tergitol™ XD (Surfactant from Dow Chemical Corp.). The ingredients were blended until homogenous.

Formulation G: The following formulation was prepared to test insect repellency (% by weight): 10% oleic acid, 109% methyl anthranilate, 3.2% Agnique® ABS 60CB (Calcium Dodecyl Benzene Sulfonate from Cognis Corporation), Agnique® SMO-20 (Sorbitan monooleate from Cognis Corporation), 2.47% AgniqueX® BP4-3103 (EO/PO block co-polymer from Cognis Corporation), 72.89% Aromatic 100 solvent. The ingredients were blended until homogenous.

Formulation H: The following formulation was prepared to test insect repellency (% by weight): 309% oleic acid, 10% methyl anthranilate, 60% Polyethylene glycol. The ingredients were blended until homogenous.

Formulation I: The following formulation was prepared to test insect repellency (% by weight): 3% oleic acid, 1% methyl anthranilate, 96% Polyethylene glycol. The ingredients were blended until homogenous.

### Example 1

Yellowjacket, Hornet and Ant Repellency Using Oleic Acid and Methyl Anthranilate Mixtures

The following formulations were prepared to test insect repellency (% by weight):

1) 3% oleic acid, 1% methyl anthranilate, 96% Sunspray 6E oil (Sunoco Oil Company) (Formulation 1)
2) 3% oleic acid, 97% Sunspray 6E oil
3) 1% methyl anthranilate, 99% Sunspray 6E oil
4) 1% oleic acid, 0.3% methyl anthranilate, 98.7% Sunspray 6E oil (Formulation D)
5) 1% oleic acid, 99% Sunspray 6E oil
6) 0.3% methyl anthranilate, 99.7% Sunspray 6E oil

Eight six-inch paper plates were baited with 6 mL of honey and two tablespoons of canned mackerel each. Two baited plates were sprayed with 0.25 mL of formulation 1 (Formulation I), two were sprayed with formulation 2, two were sprayed with formulation 3 and two were designated as controls and were left untreated. The plates were placed in a wooded area in Ewing Township, N.J., USA separated from each other by 25 yards. The plates were observed for two hours during which time German Yellowjacket wasps (Vespula germanica), bald-faced hornets (Vespula maculate), and several ant species visited and fed from the control plates but avoided all the treated plates. At the end of two hours the number of Black Carpenter ants (Camponotus pennsylvanicus) on each plate was counted, the results are summarized in Table 1A and 1B below. Only Carpenter ants were counted as they tended to chase some of the other ant species away. Also, the yellowjackets and hornets were not found on or near the treated plates however, they visited the controls to feed but frequently flew away.

Table 1A

Carpenter Ants Found on Baited Plates Test 1

TABLE 1A

| Formulation Number | Number of Ants on plates |
|---|---|
| 1 | 2 |
| 2 | 77 |
| 3 | 51 |
| Control | 68 |

Table 1B

Carpenter Ants Found on Baited Plates Test 2

TABLE 1B

| Formulation Number | Number of Ants on plates |
|---|---|
| 4 | 5 |
| 5 | 42 |
| 6 | 18 |
| Control | 66 |

The % inhibition of treated plates was calculated using the following formula: % Inhibition=(ants on control plates−ants on treated plates/ants on control plates)×100. The % inhibition for each formulation is summarized in Table 2 below.

TABLE 2
% Inhibition

| Formulation Number | % Inhibition on Treated Plates |
|---|---|
| 1 | 97% |
| 2 | −13% |
| 3 | 25% |
| 4 | 92% |
| 5 | 36% |
| 6 | 73% |

The presence of a synergistic effect between the two active ingredients is established with the aid of the Colby equation (see Colby, S. R., “Calculating Synergistic and Antagonistic Responses of Herbicide Combinations”, Weeds 1967, 15, pg 20-22): E=X+Y−(XY/100).

Using the method of Colby, the presence of a synergistic interaction between two active ingredients is established by first calculating the expected activity, ‘E’, of the mixture based on activities of the two components applied alone. If ‘E’ is lower than the observed activity, synergy is present. In the equation above, ‘X’ is the percentage control observed when oleic acid applied alone at rate ‘x’. The ‘Y’ term is percentage control observed when methyl anthranilate applied alone at rate ‘y’. The equation calculates ‘E’, the expected activity of the mixture of ‘X’ at rate ‘x’ with ‘Y’ at rate ‘y’ if their effects are strictly additive and no interaction has occurred.

For Test 1 the calculated “E” value was 15% whereas the actual value was 97%, an improvement of 82% over the expected additive effect. The “E” value for Test 2 was 83% whereas the actual value was 92%, an improvement of 9% over the expected value. Both tests indicate a synergistic effect was obtained using a 3:1 ratio of oleic acid and methyl anthranilate.

### Example 2

Bee Repellency Using an Oleic Acid and Methyl Anthranilate Mixture

Two six-inch paper plates were baited with 6 mL of honey each. One of the baited plates was sprayed with about 0.25 mL of Formulation A and one was designated as a control and was untreated. The plates were placed in a wooded area in Yardley, Pa. separated from each other by one foot, about 35 yards from the hive. The plates were observed for two hours during which time over 70 honey bees (Apis mellifera) visited and fed from the control plate but no bees neared the treated plate. At the end of two hours the number of honey bees on each plate was counted, the control had 14 bees and the treated plate had none.

### Example 3

Insect Repellency Using an Oleic Acid and Methyl Anthranilate Mixture

Two six-inch paper plates were baited with a tablespoon of canned cat food each. One of the baited plates was sprayed with 0.302 grams of Formulation F and one was designated as a control and was untreated. Honey (25 mL) was diluted with 75 mL of warm tap water and the mixture was stirred to form a homogenous solution. The plates were placed in a wooded area in Ewing Township, N.J., USA separated from each other by 5 yards. Fifty mL of the diluted honey solution was poured onto each test plate. The plates were observed at two, four and six hours for insects. At the end of two hours the number of insects on each plate was counted, the control had 7 ants and 6 German Yellowjacket wasps, the treated plate had none. At the four hour check the bait on the control plate was totally consumed and the bait was replaced, the treated plate had two ants and a dead house fly (these were removed). At six hours the control plate had 8 German Yellowjacket wasps and 4 ants where the test plate had no insects on it.

### Example 4

Wasp Repellency Using an Oleic Acid and Methyl Anthranilate Mixture

An eight inch paper plate was baited with an aqueous 25% honey solution and was placed on a cement block in a wooded area in Ewing Township, N.J. USA. After four hours about 50 German Yellow jacket wasps were actively flying around and feeding from the baited plate. When five to eight wasps were feeding on the bait, about 5 mL of Formulation F was applied as a mist over the plate and the area adjacent to the plate using a hand-pump spray bottle. All Yellowjacket wasps evacuated the plate and vicinity. One wasp died by direct contact with the spray. Many of the wasps approached to within about six inches of the treated area but no closer. Within ten minutes, all wasps were gone. After one hour one wasp was flying around the plate but did not land on or near the plate. No additional insect species were observed on or near the plate post treatment.

### Example 5

European Paper Wasp Repellency Using an Oleic Acid and Methyl Anthranilate Mixture

An active European paper wasp (Polistes sp) nest was located outside an office building in Ewing, N.J., USA having over 70 cells and about 10 wasps tending the nest. Formulation A was poured into a 32 ounce Zep® spray bottle with the nozzle adjusted to deliver a fine mist and the nest was sprayed using two pumps from the spray bottle. There were four wasps on the nest at the time of application and were killed instantly (these were removed from the nest). Within minutes three wasps were observed returning to the nest however they were repelled and would not land on the nest and finally abandoned the nest after several attempts to land. After 16 hours, two weeks and four weeks the nest was still abandoned.

### Example 6

Insect Repellency Using an Oleic Acid and Methyl Anthranilate Mixture

Formulation A was poured into a plastic spray bottle and was sprayed onto 20, eight inch paper plates, one squeeze of the pump trigger per plate. The treated plates were weighed and found that about 0.22 gm to about 0.24 gram was applied per plate. Ten treated plates were placed randomly on the ground on a farm in Sparks, Ga., USA and ten control (untreated) plates were placed on the ground about 3 feet from the treated plates. Ten treated plates were placed about 4 feet from active Red imported fire ant mounds and ten control plates were placed near the same mounds no closer than 3 feet from the treated plates. Each plate received approximately 15 mL of an aqueous 25% honey solution. The test plates were observed for four hours.

Of the randomly placed treated plates seven had no insect activity, one had two species of fly (Black fungus gnat (Bradysia sp), and Long-legged fly (Dolichopus sp), all dead) one had a wood cockroach (dead) (Parcoblatta pennsylvanica) and one had one Black fungus gnat (dead). The control plates had several ant species as well as Black fungus gnat, Long-legged fly, hover fly (Syrphidae sp), Chalcid wasp (Chalcidoidea sp), honey bee and cockroach. None of the insects on the control plates were dead.

Of the treated plates placed near active Red imported fire ant (Solenopsis invicta) mounds, none had any insect on or near them. Fifty percent of the control plates had active fire ant feeding within 15 minutes and 100% of the control plates had active fire ant feeding within 30 minutes. None of the treated plates had active fire ant feeding for four hours.

In a separate trial, plates treated with methyl anthranilate only (about 100 mg), baited with a honey solution, placed within 4 feet of fire ant mounds did not deter fire ants from feeding upon these treated plates.

A number of ant species were identified as feeding on control plates but not on the treated plates in the above examples 2, 3, 4 and 6 and in other tests conducted similarly to the above examples. Within the Formicidae family. Genera include the following:

### 1) Tetramorium—Pavement ant

### 2) Camponotus—Carpenter ant

3) Wasmannia—Little fire ant

### 4) Tapinoma—Ghost ant

5) Stenamma—Cold tolerant ant

### 6) Formica—Wood ant

7) Pachycondyla—Asian needle ant

### 8) Dorymyrmex—Odorous ant

### 9) Brachymyrmex—Rover ant

### 10) Solenopsis—Fire ant

### 11) Pheidole—Bigheaded ant

### 12) Linepithema—Argentine ant

### Example 7

Insect Repellency Using an Oleic Acid and Methyl Anthranilate Mixture

A plastic hummingbird feeder was filled with hummingbird food solution and was hung in a residential back yard and observed for two hours. The feeder was visited by 17 honey bees and 3 humming birds, all of which fed from the feeder. The feeder was sprayed with Formulation A using a plastic spray bottle and was re-hung. The treated feeder was observed for two hours and in the first 30 minutes about 25 honey bees flew close to the feeder, two coming within one inch but not landing. After 30 minutes, bees completely abandoned the feeder. During the two hour period there were 4 feeding hummingbird visits indicating that hummingbirds are not affected by the oleic acidimethyl anthranilate mixture.

### Example 8

Insect Repellency Using an Oleic Acid and Methyl Anthranilate Mixture at a Staged Picnic Setting

A picnic setting was prepared in a wooded area in Ewing, N.J., with two gallon pails, one treated and one non-treated (trash buckets), paper placemats with paper plates on them atop of a sheet of plywood placed on the ground, one placemat treated and on non-treated (picnic table setting) and open plastic trash bags, one treated and one non-treated (simulated garbage bags). The inside of one of the two gallon pails was sprayed with Formulation A. A paper cup was placed on the bottom of the pail and a paper plate baited with 10 mL of honey diluted in 20 mL of tap water was placed on the cup. The control pail was baited in the same way. One mL of Formulation A was wiped onto a paper placemat, approximately 12 inches by 15 inches. The treated placemat and a control placemat (no treatment) were placed on top of a sheet of plywood set on the ground separated by about 4 feet. A paper plate baited with a blend of canned cat food and honey was placed in the center of each placemat. The inside of a one gallon Ziploc® plastic storage bag was sprayed with about 0.5 mL of Formulation A to which about 50 mL of a dilute aqueous 25% honey solution was added. The bag was suspended from a tree branch about 4 feet from the ground using a metal coat hanger as a frame to hold the bag in an open position. A similar untreated bag was prepared as a control and placed about 10 yards apart. The site was observed for insect activity at 2 and 4 hours after set up. The observations are summarized in the Table 3 below.

TABLE 3
Insect Observations 2 and 4 Hours After Picnic Test Set Up

| | |
|---|---|
| 2 Hour | 4 Hour |

Plate in pail

| | | |
|---|---|---|
| Untreated | 10 wasps* | All bait consumed |
| Treated | No insects | 1 wasp |

Plate on placemat

| | | |
|---|---|---|
| Untreated | 10 ants, no wasps | 16 carpenter ants, 1 wasp |
| Treated | 1 carpenter ant, no wasps | 4 carpenter ants, 1 wasp |

Plastic storage bag

| | | |
|---|---|---|
| Untreated | 6 wasps | 6 wasps |
| Treated | No insects | No insects |

*Wasps observed were German Yellowjacket wasps.

The data from this test indicated that oleic acid/methyl anthranilate mixtures can be used to repel insects from a picnic or outdoor social setting.

### Example 9

Insect Repellency Using Oleic Acid and Methyl Anthranilate Mixtures

Paper plates were baited with two tablespoons of canned cat food and about 5 mL of honey solution. The plates were over-sprayed with test Formulations A, B, C, D and E, two plates per formulation. Two baited plates were left untreated as controls. The baited plates were placed in a wooded area of Ewing, N.J. After two hours the plates were observed for insect activity. The observations are summarized in the table below. Although German Yellowjacket wasps were drawn to the control plates, none were seen on any of the treated plates.

TABLE 4
Summary of Carpenter Ant observations
on Treated and Untreated Baited Plates

| | Average Number of Carpenter |
|---|---|
| | Ants (Camponotus sp) |
| Formulation Used | Observed |
| A | 0 |
| B | 14 |
| C | 29 |
| D | 33 |
| E | 89 |
| Control | 62 |

This data indicated that all rates of oleic acid and methyl anthranilate tested were repellant to wasps and the mixtures containing 400/o, 10%, 4% and 1.2% of the oleic acid/methyl anthranilate ingredients exhibited repellency to carpenter ants.

### Example 10

Insect Repellency Field Test Using Oleic Acid and Methyl Anthranilate Mixture

Formulation F (34.9 grams, was sprayed onto a 3 foot by 3 foot area of blooming goldenrod. One replicate test was also performed. Two untreated 3 foot by 3 foot areas of blooming goldenrod were included in this test. The treated and untreated areas were observed for one hour after treatment and also between 2 and 3 hours after treatment. The insects observed in these test areas are summarized in the table below.

TABLE 5
Summary of Insects Observed in or on Blooming Goldenrod

| Control Area | Treated Area |
|---|---|
| Hour 0-1 | Hour 0-1 |
| 8 Honeybees; 5 flies (Mixed Species); 1 Potter | No Insect Activity |
| Wasp (Eumeninae fraternus); 1 Tawny Skipper | |
| Moth (Polites themistocles) | |
| Hour 2-3 | Hour 2-3 |
| 7 Honeybees; 6 flies (Mixed Species); 2 | No Insect Activity |
| Tawny Skipper Moths; 1 Corn Rootworm | |
| Beetle; 1 Jumping Spider (Salticus sp) | |

This data indicated that the oleic acid and methyl anthranilate mixture has a repelling effect when sprayed onto blooming goldenrod.

### Example 11

Honey Bee Repellency Using Oleic Acid and Methyl Anthranilate Mixtures

Ten paper plates were baited with about 5 mL of honey. Formulations A, B, C, D and E were diluted with tap water 1 part formulation to 10 parts of water. Baited plates were over-sprayed with the diluted formulations, one plate per formulation. A treated and untreated plate was placed on a table about 35 yards from a honeybee hive. Each test rate was observed for a period of time and observations of honeybees on the test plates made every few minutes. After each observation the plates positions were switched to eliminate positional effects caused by the bees favoring certain locations of the plates. The lowest rate was tested first (Formulation E) and the highest rate (Formulation A) was tested last. The summary of honeybee on each plate is summarized in the table below.

TABLE 6
Honeybees Observed on Baited Test Plates

| | | Number of | Number of |
|---|---|---|---|
| Test | Elapsed Tune From | Honeybees on | Honeybees on |
| Formulation | Start (minutes) | Control Plate | Test Plate |
| E | 5 | 6 | 0 |
| E | 63 | 19 | 0 |
| E | 64 | 7 | 2 |
| E | 67 | 7 | 7 |
| E | 70 | 7 | 10 |
| E | 89 | 0 | 15 |
| E | 94 | 3 | 21 |
| E | 100 | 19 | 1 |
| D | 5 | 3 | 1 |
| D | 10 | 10 | 2 |
| D | 42 | 2 | 4 |
| D | 55 | 4 | 5 |
| D | 69 | 9 | 6 |
| D | 90 | 12 | 5 |
| D | 122 | 10 | 6 |
| D | 137 | 12 | 8 |
| C | 5 | 0 | 3 |
| C | 14 | 9 | 6 |
| C | 24 | 8 | 5 |
| C | 39 | 12 | 12 |
| B | 5 | 0 | 0 |
| B | 9 | 4 | 0 |
| B | 30 | 13 | 2 |
| B | 40 | 9 | 1 |
| B | 49 | 16 | 1 |
| B | 54 | 12 | 0 |
| A | 5 | 0 | 0 |
| A | 10 | 14 | 0 |
| A | 11 | 19 | 1 |
| A | 14 | 19 | 0 |
| A | 16 | 22 | 0 |
| A | 24 | 18 | 0 |
| A | 27 | 21 | 0 |
| A | 36 | 17 | 1 |
| A | 43 | 20 | 0 |

As can be seen from the above data, the test solutions repelled honeybees from treated baited test plates.

### Example 12

Eastern Subterranean Termite Repellency

A termite test arena was prepared by placing a piece of clear Plexiglas (about 8 inches by 12 inches) flat on a table (bottom piece of Plexiglas). Three square pieces of corrugated cardboard (about 2 inches by 2 inches) were laid on the bottom Plexiglas, one centered at the bottom edge and one at each top edge right and left side, with the grooves running vertical. These cardboard pieces will provide food for the termites during the test. Water 30 grams was added to 300 grams of natural play sand and the mixture stirred well to thoroughly incorporate the water. A piece of Plexiglas, approximately 1 inch wide was placed on the bottom piece of Plexiglas touching the lower edges of the cardboard pieces located at the top right and left edges. The wetted sand was poured around all the cardboard pieces. The 1 inch wide piece of Plexiglas was removed leaving a 1 inch gap. This gap was filled with ground up chalk, about 30 grams treated with 3 mL of Formulation H and 3 mL of distilled water. A top piece of Plexiglas was placed on top of the sand, chalk and cardboard, this top piece having a 1 inch diameter hole that is positioned over the bottom piece of cardboard, and the bottom and top pieces of Plexiglas clamped together with binder clips. About 200 worker Eastern subterranean termites (Rhinotermitidae: Reticulitermes flavipes) were placed onto the cardboard through the opening in the top piece of Plexiglas. The hole was covered with a plastic Petri dish cover and taped to the Plexiglas. The test arena was stored in a loosely closed black plastic bag at ambient temperature. An untreated control arena was also included in the evaluation. The test and control arenas were examined daily for six days. At day 6 the untreated control arena had termite tunnels through the chalk layer and feeding damage to all pieces of cardboard. The treated arena had termite tunnels up to the treated chalk layer but no tunnels through the chalk layer; feeding damage was limited to the bottom piece of cardboard, indicating a repelling effect.

### Example 13

German Cockroach Repellency Assay

A cockroach test arena was prepared by coating the upper edges of a 12 inch wide by 20 inch long by 3 inch high polystyrene tray with a 50/50 mixture of petroleum jelly to keep the cockroaches from escaping. Two sections were cut from a bottom of a cardboard egg carton and inverted to form a cockroach harborage. One section was sprayed with Formulation H and placed at one end of the test arena. The second section was not treated and placed at the opposite end of the arena Three Smarties® roll candies were placed at the midpoint of the arena as a food source. Fifty adult male German cockroaches (Blattella germanica) were placed in the center of the test arena and observed daily for 9 days. This test was run in duplicate. The observation results of which harborage the cockroaches preferred are summarized in the following table.

TABLE 7
German Cockroach Repellency Results

| | Number Under Untreated | | Number Under Treated | |
|---|---|---|---|---|
| Day of | Harborage | | Harborage | |
| Observation | Replicate 1 | Replicate 2 | Replicate 1 | Replicate 2 |
| 1 | 10 | 27 | 5 | 4 |
| 2 | — | — | 1 | 0 |
| 3 | 27 | 28 | 5 | 5 |
| 4 | 18 | 15 | 3 | 7 |
| 5 | — | — | 4 | 1 |
| 6 | 17 | 16 | 8 | 8 |
| 7 | 12 | 16 | 6 | 3 |
| 8 | 28 | 33 | 1 | 12 |
| 9 | 19 | 24 | 4 | 15 |

As can be seen from the data above, German cockroaches preferred the untreated harborage over the treated harborage, therefore exhibiting repellency.

### Example 14

Brown Marmorated Stinkbug Repellency

A test arena was prepared by coating the upper edge of a Tupperware® 6 inch by 8 inch storage container with a 50/50 mixture of petroleum jelly and mineral oil to prevent stinkbugs from escaping. A green bean was dipped into Formulation F and was placed at one end of the arena. An untreated green bean was placed at the other end of the container. Fourteen Brown marmorated stinkbugs (2^{nd} to 4^{th} instar, Pentaiomidae: Halyomoropha halys) were placed into the center of the arena. A second test arena was prepared as a control using untreated green beans at each end of the container. The test arenas were maintained at ambient temperature for 96 hours, at which time it was observed that in the second test arena (control) stinkbugs fed off both green beans equally while the stinkbugs in the container with the treated green bean fed on the untreated green bean only indicating a repelling effect.

### Example 15

Repelling Tobacco Bud Worm Moths Egg Deposition on Cotton

Four cotton plants (non BT cotton plants with two true leaves) were treated with Formulation F by placing a single drop of the formulation onto the top surface of each leaf. A treated plant was placed at one side of a BioQuip® Products wire mesh cage (14″ by 14″ by 14″); an untreated cotton plant was placed at the other end of the cage. There were four replicates. Five gravid female tobacco budworm moths (Geometricdae helicoverpa) were introduced into each cage. Sixteen hours after moth introduction the number of eggs deposited on the treated and untreated cotton leaves were counted. The table below summarizes the egg count in the four replicate tests.

TABLE 8
Tobacco Budworm Moth Egg Deposition on Cotton Leaves

| | Number Of Eggs Per Plant | | | | |
|---|---|---|---|---|---|
| Treatment | Rep 1 | Rep 2 | Rep 3 | Rep 4 | Average |
| Formulation F | 3 | 2 | 2 | 7 | 3.5 |
| Control | 27 | 37 | 37 | 54 | 38.8 |

This data indicates that plants treated with oleic acid and methyl anthranilate provide a repellency effect to Tobacco budworm moths when selecting oviposition sites.

### Example 16

Repelling Brown Dog Ticks

Formulations H and I were tested for repellency of Brown dog ticks (Rhipicephalus sanguineus) in the following manner:

Five strips of filter paper, 1 inch by 3 inches, were each treated with 1 mL of the test. Formulation and were allowed to dry. Untreated strips of filter paper of the same dimensions were stapled to the bottom of each treated strip providing a final test strip 6 inches long. The test strips were suspended vertically over a tray with the untreated portion at the bottom. An additional five test strips were prepared and were untreated to be used as controls. Five mixed sex dog ticks were tested per replicate. One tick at a time was introduced to the untreated part of the test strip and allowed to quest upwards. Ticks were observed for up to one minute to determine if the tick crossed onto the treated filter paper and continued to crawl upward. Any tick that stopped, turned around or dropped off after contacting the treated portion of the test strip was classified as repelled. All 25 ticks were repelled when Formulation H and Formulation I were tested. None of the ticks were repelled in the control tests. These tests indicate that formulations of oleic acid and methyl anthranilate are excellent repellants of Brown dog ticks.

### Example 17

Repelling Yellow Fever Mosquito

Formulations H and I were tested for repellency of Yellow Fever mosquito (Aedes aegypti) in the following manner:

A mosquito exposure container and membrane feeder was used to present mosquitos with a choice of three bovine blood-filled wells covered with a collagen membrane. The blood was prepared by adding 72 mg of ATP disodium salt to 26 mL of bovine blood as a feeding stimulant. The blood was poured into the wells of the membrane feeder until each well was completely filled (meniscus slightly above the level of the well. Circular pieces of collagen (3 cm in diameter) were placed over each well. One membrane was treated with 25 microliters of Formulation H, one with 25 microliters of Formulation I and one was untreated. Five replicates were tested. The membrane feeder was connected to a heated water bath and warmed to 37° C. to 40° C. The exposure container with 250 female mosquitoes was placed over the feeder and opened and the number of mosquitoes probing the membrane covering each well was recorded every two minutes for a period of 20 minutes. A new batch of female mosquitoes was used for each replicate. The number of probes per replicate was totaled and the totals were averaged. The average number of probes on the control membrane was 65, the average probes on the Formulation H membrane was 0, and the average probes on the membrane treated with Formulation I was 6.8. This data shows that formulations of oleic acid and methyl anthranilate are excellent repellants of Yellow Fever mosquitoes.

### Example 18

Repelling Cat Flea LarvaFormulation H and dilutions thereof were tested for repellency to Cat flea larvae (Ctenocxephalides felis) in the following manner:

Test solutions of Formulation F were prepared by diluting Formulation F with polyethylene glycol. Formulation F contains 30% by weight oleic acid and 10% by weight methyl anthranilate (40% total active ingredients); the first dilution provided a concentration of 12% active ingredients (Formulation 18-1); the second dilution provided a concentration of 4% active ingredients (Formulation 18-2); the third dilution provided a concentration of 1.2% active ingredients (Formulation 18-3); and the final dilution provided a concentration of 0.4% active ingredients (Formulation 18-4).

A round piece of black construction paper (150 mm diameter) was treated with test solutions of Formulation H by covering half of the paper with aluminum foil prior to spraying the test formulation onto the uncovered half. The treated paper was affixed to the bottom of a 150×15 mm Petri dish base. The Petri plate was allowed to dry in a laboratory fume hood for several minutes. Ten cat flea larvae were released in the median area of the treated paper and were observed to be on the untreated or treated portion of the test paper for 2 to 3 hours. The observations are summarized in the table below.

TABLE 9
Cat Flea Larvae Repellency

| Formulation | Elapsed | Number Of Larvae in | Number Of Larvae in |
|---|---|---|---|
| Treatment | Time (Hr) | Untreated Area | Treated Area |
| F | 1 | 10 | 0 |
| | 2 | 10 | 0 |
| | 2.5 | 10 | 0 |
| 18-1 | 1 | 10 | 0 |
| | 3 | 9 | 1 |
| 18-2 | 1 | 10 | 0 |
| | 3 | 10 | 0 |
| 18-3 | 1 | 10 | 0 |
| | 3 | 10 | 0 |
| 18-4 | 1 | 10 | 0 |
| | 3 | 10 | 0 |

This data indicates that cat flea larvae are repelled by formulations containing oleic acid and methyl anthranilate at rates as low as 0.4% of the combined active ingredients.

### Example 19

Southern Corn Rootworm Repellency

Test A: A test arena was prepared by cutting a piece of round filter paper in half and placing both halves on the bottom of a plastic Petri dish leaving a small gap between the two halves of filter paper. The pieces of filter paper were moistened with 1 mL of deionized water per side. Three germinated corn seedlings were dipped into Formulation F, allowed to dry, and placed at one end of the Petri dish on one piece of filter paper as far as possible from the gap between the pieces of filter paper. Three untreated germinated corn seedlings were placed on the other piece of filter paper as far as possible from the gap. Eighteen second instar Southern corn rootworms (Diabrotica undecimpunctata howardi) were placed in the midline gap between the two pieces of filter paper and a plastic Petri lid was placed on top. An untreated control having three untreated germinated corn seedlings at each side was tested as well as a fully treated test where all six germinated corn seedlings were included in the test. The Petri dishes were maintained at ambient temperature and humidity in a dark growth chamber for 24 hours. Observations were made at 4 hours and 24 hours to determine if the larvae were feed in the germinated corn seedlings. These observations are summarized in the table below.

TABLE 10
Southern Corn Rootworm Larvae Feeding Observations

| Treatment | Larvae Feeding at 4 Hours | Larvae Feeding at 24 Hours |
|---|---|---|
| Untreated vs | 9 larvae on untreated corn | 12 larvae on untreated corn |
| Treated | 0 larvae on treated corn | 0 larvae on treated corn |
| | remainder in middle | remainder in middle |
| Untreated | 6 larvae on each side (12 | 6 larvae on one side |
| Control | total) | 12 larvae on the other side |
| | remainder in middle | remainder in middle |
| Both Sides | 0 feeding | 1 larvae feeding on corn |
| Treated | All in middle or on edge | remainder in middle or edge |
| | of filter paper | of filter paper |

This data indicates that Southern corn rootworm larvae are repelled from feeding by formulations containing oleic acid and methyl anthranilate.

Test B: A 50 mL polypropylene conical tube was filled with 20 mL of top soil containing 10% water. About 75 Southern corn rootworm eggs (Diabrotica undecimpunctata howardi) were placed onto the soil. The eggs were suspended in distilled water and transferred to the soil with a pipette. A final layer of 10 mL of top soil, for untreated control tests or treated soil for treated tests was added to the top. For treated tests, 375 mg of Formulation F was diluted with 15 mL of distilled water. This solution was added to 150 mL of top soil and mixed thoroughly. Treatments in which treated soil was used for both the bottom layer and top layer were also included. A 1 inch lettuce disk was placed on the top of top layer of soil as bait, the tubes were capped and stored at ambient temperature and humidity for 7 days. The lettuce disks were replaced after 3 days. After 7 days the larvae that had hatched and reached the lettuce disks were counted as well as observing and larvae that had hatched and remained in the soil. Each test was performed in duplicate. The observations made are summarized in the table below.

TABLE 11
Southern Corn Rootworm Egg Hatch
and Larvae Feeding Observations

| Treatment | Replicate | Observation |
|---|---|---|
| Untreated control | 1 | 44 larvae on lettuce disk |
| | 2 | 63 larvae on lettuce disk |
| Untreated bottom soil, | 1 | 0 on lettuce disk |
| treated top soil | | Very few eggs hatched |
| | 2 | 0 on lettuce disk |
| | | Very few eggs hatched |
| Treated bottom soil, treated | 1 | 0 on lettuce disk |
| top soil | | 0 eggs hatched |
| | 2 | 0 on lettuce disk |
| | | 0 eggs hatched |

This data indicates that Southern corn rootworm larvae are repelled from feeding by formulations containing oleic acid and methyl anthranilate and that Southern corn rootworm eggs fail to hatch in the presence of oleic acid and methyl anthranilate.

### Example 20

Saw-Toothed Grain Beetle Repellency

A round piece of black construction paper (150 mm diameter) was treated with Formulation H by covering half of the paper with aluminum foil prior to spraying the test formulation onto the uncovered half. The treated paper was affixed to the bottom of a 150×15 mm Petri dish base. The Petri plate was allowed to dry in a laboratory fume hood for several minutes. Fifty Saw-toothed grain beetles (Oryzaephilus surinamensis) were released in the median area of the construction paper and were observed to be on the untreated or treated portion of the test paper for several hours. A control test in which one side of the construction paper was sprayed with polyethylene glycol only was also included. The observations are summarized in the table below.

TABLE 12
Saw-toothed Grain Beetle Repellency Observations
Saw-Toothed Gram Beetles Observed on Treated or Untreated Paper

| | | |
|---|---|---|
| Elapsed Time (Hr) | Treated | Untreated |

Formulation H treatment

| | | |
|---|---|---|
| 1 | 5 | 45 |
| 2 | 6 | 44 |
| 3 | 3 | 47 |
| 4.5 | 2 | 48 |
| 5.5 | 2 | 48 |
| 6.5 | 11 | 39 |
| 21.5 | 12 | 38 |

Control (Polyethylene glycol = Treated)

| | | |
|---|---|---|
| 1 | 25 | 25 |
| 2 | 25 | 25 |
| 3 | 30 | 20 |
| 18.5 | 40 | 10 |

This data indicates that a mixture of oleic acid and methyl anthranilate exhibit repellency to Saw-toothed grain beetles.

### Example 21

Confused Flour Beetle Repellency

A round piece of black construction paper (150 mm diameter) was treated with Formulation H by covering half of the paper with aluminum foil prior to spraying the test formulation onto the uncovered half. The treated paper was affixed to the bottom of a 150×15 mm Petri dish base. The Petri plate was allowed to dry in a laboratory fume hood for several minutes. Thirty Confused flour beetles (Tribolium confusum) were released in the median area of the construction paper and were observed to be on the untreated or treated portion of the test paper for several hours. A control test in which one side of the construction paper was sprayed with polyethylene glycol only was also included. The observations are summarized in the table below.

TABLE 13
Confused Flour Beetle Repellency Observations
Confused Flour Beetles On Treated or Untreated Paper

| 10:30 am start | Formulation H | | Polyethylene Glycol (Control) | |
|---|---|---|---|---|
| Time (Hr) | Treated | Untreated | Treated | Untreated |
| 1 | 1 | 29 | — | — |
| 2 | 0 | 30 | — | — |
| 3 | 0 | 30 | — | — |
| 4 | 0 | 30 | 25 | 25 |
| 5 | 0 | 30 | 25 | 25 |
| 6 | 2 | 28 | 30 | 20 |
| 21.5 | 2 | 28 | 40 | 10 |

This data indicates that a mixture of oleic acid and methyl anthranilate exhibit repellency to Confused flour beetles.

### Example 22

Small Carpenter Bee Repellency

A residential area that was heavily infested with small carpenter bee nesting holes in the ground (Apidae: Ceratina sp.) was marked off in 4 two foot by two foot test plots. Each test plot contained 24 or more small carpenter bee nesting holes. One of the test plots was treated with Formulation F, one with Formulation 18-1 and one with Formulation 18-2. The remaining test plot was left untreated as a control. The formulations were applied using a hand held spray bottle spraying about 12 inches above the test plot, approximately 24 spray pumps per test plot. The test plots were observed for 8 hours to determine if the small carpenter bees entered the nesting holes or were repelled. The test was performed on a warm day in May 2013. The table below summarizes the observations.

TABLE 14
Small Carpenter Bee Repellency Observations

| | Observation Time | | | | | | | |
|---|---|---|---|---|---|---|---|---|
| | 10 | 11 | 12 | | | | | 5 |
| Treatment | AM | AM | PM | 1 PM | 2 PM | 3 PM | 4 PM | PM |
| Control | | | | | | | | |
| No. Bees | 3 | 4 | 3 | 11 | 4 | 4 | 1 | 0 |
| entering | | | | | | | | |
| Hole | | | | | | | | |
| No. Bees | 3 | 7 | 6 | 9 | 11 | 5 | 2 | 0 |
| Flying | | | | | | | | |
| Above | | | | | | | | |
| Holes | | | | | | | | |
| Formulation | | | | | | | | |
| 18-2 | | | | | | | | |
| No. Bees | 0 | 0 | 0 | 0 | 0 | 0 | 0 | 0 |
| entering | | | | | | | | |
| Hole | | | | | | | | |
| No. Bees | 4 | 8 | 10 | 8 | 7 | 3 | 0 | 0 |
| Flying | | | | | | | | |
| Above | | | | | | | | |
| Holes | | | | | | | | |
| Formulation | | | | | | | | |
| 18-1 | | | | | | | | |
| No. Bees | 0 | 0 | 0 | 1 | 0 | 0 | 0 | 0 |
| entering | | | | | | | | |
| Hole | | | | | | | | |
| No. Bees | 2 | 5 | 5 | 10 | 8 | 4 | 1 | 0 |
| Flying | | | | | | | | |
| Above | | | | | | | | |
| Holes | | | | | | | | |
| For- | | | | | | | | |
| mulation. | | | | | | | | |
| F | | | | | | | | |
| No. Bees | 0 | 0 | 0 | 0 | 0 | 0 | 0 | 0 |
| entering | | | | | | | | |
| Hole | | | | | | | | |
| No. Bees | 2 | 2 | 5 | 7 | 9 | 2 | 1 | 0 |
| Flying | | | | | | | | |
| Above | | | | | | | | |
| Holes | | | | | | | | |

After 28 hours no bees were observed near the treated plots. This data indicates that a mixture of oleic acid and methyl anthranilate exhibit repellency to small carpenter bees.

In addition to the insects mentioned in the foregoing examples the following orders of insects have been repelled by a mixture of oleic acid and methyl anthranilate (Family, Genus):

Hymenoptera, carpenter bee (Apidae Xylocopa) and bumblebee (Apidae Bombus); Hemiptera, bed bug (Cimicidae Cimex):
Diptera, common house mosquito (Culicidae Anopheles); house fly (Muscidae Musca), flesh fly (Sarcophagidea Sarcophagi), fruit flies (Drosophilidae Drosophila), phorid flies (Phoridae Megaselia);
Lepidroptera, Indian meal moth (Pyralidae Plodia);
Araneae, Black widow spider (Theridiidae Latrodectus), Brown recluse spider (Sicariidae, Loxosceles), Wolf spider (Lycosidae), Daddy long-legs spider (Pholcidae);
Tylenchida, Root knot nematode (Meloidogtnidae Meloidogynae)

### Example 23

Assessment of Bird Repellent Activity

Bird repellent activity was tested by treating black oil sunflower seeds with Formulation F diluted by 50% with deionized water. Treatment 1 was prepared by coating black oil sunflower seed with 10 mL of the diluted formulation per kilogram of seed, Treatment 2 was prepared by coating black oil sunflower seed with 50 mL of the diluted formulation per kilogram of seed. Four 20″ by 20″ wooden trays were suspended from the cross brackets of a Duncraft® Squirrel Stopper System bird feeder and 75 grams of treated seed was placed onto two trays (designated to be used for treated seed only) and 75 grams of untreated seed was placed onto the two remaining trays as controls. The trays were monitored daily for birds feeding on the seed. After each day the remaining seed was collected for weighing and replaced with new seed. The tray positions were moved by one position every time they were refilled to eliminate positional effects caused by the birds favoring certain locations of the feeder over other positions. The tests were run for two weeks per treatment, 4 replicates per treatment. There was no statistical difference between the amount of treated seed consumed and the amount of untreated seed consumed. Birds observed feeding on the seed include Turtle dove, House finch, American goldfinch, Downey woodpecker, Harry woodpecker, Northern flicker, Red bellied woodpecker, Blue jay, Carolina chickadee. Tufted titmouse, Carolina wren, Grey catbird. European starling. House sparrow, Cardinal and Common grackle.

### Example 24

Repelling Yellow Fever Mosquito

A mixture of 3.0 grams of oleic acid, 1.0 gram of methyl anthralinate and 96.0 grams of isopropyl alcohol was stirred until homogenous. This formulation was labeled Ex. 24. For comparative purposes a 5% solution of N,N-diethyl-3-methylbenzamide in isopropyl alcohol (active ingredient in DEET Insect Repellant) was also tested. This sample was labeled 5% DEET. Isopropyl alcohol (IPA), water and untreated collagen membranes were also tested.

An in vitro laboratory trial was conducted to evaluate the repellency of a test formulation aged at various intervals against female Aedes aegypti. 250 female adult mosquitoes, Aedes aegypti, were used per replicate (5 replicates per test). Adult mosquitoes were 6 days old and were deprived of sucrose solution for approximately 18 hours prior to testing. An exposure container was used to present the mosquitoes with a choice of five blood-filled wells covered with collagen membranes. The collagen membranes were treated by pipetting 25 μL of the test formulation or solvent onto the appropriate membrane and spreading it evenly with the tip of the pipette. The membranes were left to age undisturbed at ambient temperature and humidity for 2 hours.

The exposure container used was a 30.5 cm×30.5 cm×30.5 cm rigid plastic frame supported by four, 4-cm high legs, with a sleeved entry on one side and a sliding door on the bottom.

The membrane feeder consisted of five wells (3 cm in diameter×8 mm in depth) in line on a hollow plastic block (6 cm wide×22 cm long×3 cm deep), which fits through the sliding door in the bottom of the exposure container. Hoses attached to each side of the block circulate heated water that is pumped from a water bath. The sliding door in the bottom of the exposure container covers and uncovers the wells in the membrane feeder, allowing mosquitoes to access the wells.

The membrane feeder was connected to a heated water bath, and warm water passed through the feeder via a circulating pump so that the wells were warmed to 89-95° F. Seventy-two (72) mg of ATP (disodium salt) were added to 26 mL of warmed citrated bovine blood, which was poured into the wells until they were completely full. The collagen membranes, after being treated and aged as described above, were placed over each of the wells, completely covering the blood. Care was taken to eliminate all air bubbles from between the membrane and the surface of the blood.

The mosquitoes were released into the exposure container just prior to exposure to the membranes. After the five minutes, the exposure container was placed on the membrane feeder and the sliding door opened, allowing the mosquitoes to access the wells. The number of mosquitoes probing each membrane was recorded every two minutes for twenty minutes.

The above procedures were repeated until five replicates were completed. A new batch of 250 female mosquitoes and fresh blood were used for each replicate, and the wells were cleaned in between replicates. The position of the treatments was rotated for each replicate, so that each treatment was tested on each of the five wells.

The data from the five replicates are presented below.

TABLE 15
Replicate 1

| Time (min) | Ex. 24 | 5% DEET | IPA | Water | Untreated |
|---|---|---|---|---|---|
| 2 | 0 | 0 | 1 | 1 | 0 |
| 4 | 0 | 3 | 9 | 4 | 1 |
| 6 | 0 | 5 | 8 | 7 | 3 |
| 8 | 1 | 7 | 19 | 13 | 2 |
| 10 | 2 | 10 | 12 | 15 | 9 |
| 12 | 7 | 14 | 15 | 14 | 13 |
| 14 | 8 | 16 | 15 | 16 | 15 |
| 16 | 9 | 16 | 18 | 23 | 15 |
| 18 | 6 | 15 | 18 | 16 | 17 |
| 20 | 5 | 12 | 16 | 17 | 19 |
| Total | 38 | 98 | 131 | 126 | 94 |

Replicate 2

| Time (min) | Untreated | Ex. 24 | 5% | IPA | Water |
|---|---|---|---|---|---|
| 2 | 0 | 0 | 2 | 7 | 4 |
| 4 | 12 | 1 | 5 | 13 | 6 |
| 6 | 12 | 2 | 8 | 20 | 13 |
| 8 | 14 | 3 | 6 | 18 | 15 |
| 10 | 19 | 6 | 6 | 21 | 17 |
| 12 | 27 | 13 | 6 | 11 | 16 |
| 14 | 13 | 9 | 4 | 14 | 11 |
| 16 | 16 | 9 | 5 | 15 | 10 |
| 18 | 18 | 8 | 7 | 15 | 9 |
| 20 | 21 | 6 | 8 | 11 | 9 |
| Total | 152 | 57 | 57 | 145 | 110 |

Replicate 3

| Time ( min) | Water | Untreated | Ex. 24 | 5% | IPA |
|---|---|---|---|---|---|
| 2 | 13 | 14 | 6 | 3 | 12 |
| 4 | 15 | 16 | 5 | 6 | 15 |
| 6 | 23 | 15 | 4 | 5 | 13 |
| 8 | 16 | 18 | 3 | 7 | 13 |
| 10 | 14 | 16 | 3 | 7 | 9 |
| 12 | 12 | 15 | 3 | 6 | 10 |
| 14 | 11 | 18 | 4 | 5 | 6 |
| 16 | 18 | 18 | 5 | 9 | 12 |
| 18 | 16 | 15 | 8 | 5 | 9 |
| 20 | 17 | 14 | 1 | 8 | 7 |
| Total | 155 | 159 | 42 | 61 | 106 |

Replicate 4

| Time (min) | IPA | Water | Untreated | Ex. 24 | 5% |
|---|---|---|---|---|---|
| 2 | 2 | 5 | 3 | 0 | 1 |
| 4 | 12 | 4 | 6 | 0 | 0 |
| 6 | 6 | 5 | 8 | 0 | 0 |
| 8 | 14 | 9 | 8 | 0 | 0 |
| 10 | 9 | 9 | 8 | 1 | 0 |
| 12 | 13 | 8 | 6 | 0 | 0 |
| 14 | 11 | 11 | 4 | 0 | 0 |
| 16 | 15 | 8 | 5 | 0 | 0 |
| 18 | 10 | 9 | 2 | 0 | 0 |
| 20 | 9 | 7 | 3 | 0 | 0 |
| Total | 101 | 75 | 53 | 1 | 1 |

Replicate 5

| Time (min) | 5% | IPA | Water | Untreated | Ex. 24 |
|---|---|---|---|---|---|
| 2 | 5 | 7 | 7 | 4 | 1 |
| 4 | 3 | 13 | 13 | 6 | 2 |
| 6 | 8 | 20 | 10 | 13 | 1 |
| 8 | 10 | 23 | 18 | 13 | 3 |
| 10 | 16 | 21 | 21 | 15 | 2 |
| 12 | 20 | 21 | 18 | 11 | 5 |
| 14 | 13 | 19 | 22 | 18 | 5 |
| 16 | 15 | 18 | 19 | 20 | 1 |
| 18 | 11 | 23 | 17 | 20 | 1 |
| 20 | 18 | 23 | 14 | 14 | 1 |
| Total | 119 | 188 | 159 | 134 | 23 |

Average of 5 Replicates

| Treatment | Ex. 24 | DEET | IPA | Water | Untreated |
|---|---|---|---|---|---|
| Total Probes | 32.2 | 67.2 | 134.2 | 125.0 | 118.4 |

The above results show that the composition of the present invention provides superior repellency than does DEET.

### Example 25

An esthetically pleasing and cosmetically acceptable skin care Formulation J was prepared in a lotion form in accordance to the following method.

Initially, an oil phase was prepared by combining and stirring the ingredients below heated to 60° C. (beyond the melting point of stearic acid):

| | % w/w |
|---|---|
| Methyl Anthranilate (MA) | 6.7 |
| Oleic acid (OA) | 1.3 |
| Stearic acid | 2.0 |
| Rhodasurf L-9 | 3.0 |

The aqueous phase was then prepared by mixing the ingredients below heated to 60° C. Rhodasurf L-9 is the Rhodia brand of ethoxylated C12-C15 alcohols. Other vendor surfactants are also acceptable

| | % w/w |
|---|---|
| Water | 75.0 |
| Glycerin | 5.0 |

Once both phases were prepared and heated to 60° C., they were combined with shear until emulsified lotion was formed. Milder stirring was commenced and the product was allowed to cool to 25° C. The lotion formulation formed was stable at 25° C.

### Example 26

Formulas K and L were prepared according to the following recipe:

| Ingredient | Formulation K | Formulation L |
|---|---|---|
| Aqueous phase: | | |
| Water | 50 | 50 |
| Lecithin | 6 | 6 |
| Organic Phase: | | |
| Oleic acid (OA) | 1.6 | 0.8 |
| Methyl anthranilate (MA) | 6.4 | 3.2 |
| Caproyl 90 | q.s | q.s |

These formulas were prepared by separately formulating the aqueous and oil phases. Aqueous phase was prepared by dissolving lecithin into water and heating to 80° C. Subsequently the organic phase was prepared by dissolving the OA and MA into Caproyl 90™ (propylene glycol monocaprylate type II NF) and heating to 80° C. Under high shear the organic phase was added to the aqueous phase and high shear was continued until particle size reaches 3 microns. Next, heating was discontinued and mild stirring commenced and continued until the lotion reached 25° C., at which time the product was packaged and tested for efficacy against biting pests.

The preliminary toxicity tests indicated favorable ophthalmic, dermatologic and oral toxicity profile. The efficacy of the lotion formulation was assessed and the results indicated favorable are depicted below:

TABLE 16

| Formula | % efficacy |
|---|---|
| 4% + 20% Isopropyl myristate (“IPM”) | 99.2 |
| 4% = 0.8% oleic acid + 3.2% methyl anthranilate (MA) | |
| 8% + 20% IPM | 99.7 |
| 8% = 1.6% oleic acid + 6.4% MA | |
| 4% + 10% IPM* | 99.5 |
| 4% = 0.8% oleic acid + 3.2% MA | |
| 8% + 10% IPM | 99.8 |
| 8% ⁼ 1.6% oleic acid + 6.4% MA | |
| 8% Lotion (Formulation J) | 99.7 |
| 4% no IPM | 87.7 |
| 4% = 0.8% oleic acid + 3.2% methyl anthranilate | |

The lotions were superior in both efficacy and toxicity profile as compared to 20% Deet.

### Example 27

Additional skin care Formulations M, N, O in the form of body spray containing repellent mixtures were prepared containing the ingredients listed below:

These formulas may contain sunscreen, to further protect subjects not only against insect attack but also the harmful solar rays. Preferably, these additional formulas are clear, low viscosity body sprays.

In preparing Formulation M, phases A, B and C were prepared separately. Phase A was heated to 80° C. until all ingredients are dissolved. Phase A was then cooled down to room temperature and mixed with respectively phase B and then phase C while stirring. These examples further define formulations of Necromone Manufacturing Use Product (or MUP), in all cases defined for the examples as 1 part oleic acid to 4 parts methyl anthranilate.

% w/w

Phase A

| | |
|---|---|
| Dub DOA (Stearinerie Dubois Fils) (Diethylhexyl adipate) | 13.00 |
| Eusolex HMS (Merck) (Homosalate) | 15.00 |
| Neo Heliopan 303 (Symrise) (Octocrylene) | 10.00 |
| Neo Heliopan OS (Symrise) (Octisalate) | 5.00 |
| Parsol 1789 (DSM) (Avobenzone) | 3.00 |
| Tinosorb S (Ciba) (Bemotrizinol) | 2.00 |

Phase B

| | |
|---|---|
| Dow Corning 345 fluid (Dow Corning) (Cyclopentasiloxane | 5.00 |
| (and) cyclohexasiloxane) | |

Phase C

| | |
|---|---|
| Necromone MUP | 8.00 |
| Ethanol 96% (Merck) | q.s. to 100% |

In preparing Formulation N, phases A and B were prepared separately. Phase A was then heated to 80° C. until all ingredients were dissolved. Phase A was then cooled down to room temperature and mixed with phase B while stirring.

Phase A

| | |
|---|---|
| Finsolv TN (Innospec) (C12-15 alkyl benzoate) | 65.20 |
| Nikkol TOC (Nikko Chemical) (Triethylhexyl | 7.00 |
| citrate) | |
| Permethyl 99A (Presperse) (Isododecane) | 17.00 |
| Necromone MUP | 8.00 |

Phase B

| | |
|---|---|
| Sylvasol 80-E70 (Arizona Chemical) (Alcohol denat. | 6.80 |
| and polyamide-3) | |

In Preparing Formulation O, phases A and B were prepared separately. Phase A was then heated to 80° C. until all ingredients were dissolved. Phase A was then cooled down to room temperature and mixed with phase B while stirring.

Phase A

| | |
|---|---|
| DI water | 42 |
| 2% Kelzan (xanthan gum) | 14 |
| Necromone MUP | 8.00 |

Phase B

| | |
|---|---|
| Sylvasol 80-E70 (Arizona Chemical) (Alcohol | 6.80 |
| denat. and polyamide-3) | |

### Example 28

Female adult Aedes aegypti mosquitoes were obtained from i2LResearch USA, Inc.'s in-house colony, 250 mosquitoes were used per replicate (5 replicates per test). Adult mosquitoes were 5-12 days old and were deprived of sucrose solution for approximately 18 hours prior to testing.

The following test substances were assessed:

TABLE 17

| | Formulation | Active ingredients |
|---|---|---|
| 1 | Formulation P | 20% IPM, 4% Necromone MUP in EtOH |
| 2 | Formulation Q | 20% IPM, 8% Necromone MUP in EtOH |
| 3 | Formulation R | 10% IPM, 4% Necromone MUP in EtOH |
| 4 | Formulation S | 10% IPM, 8% Necromone MUP in EtOH |
| 5 | Formulation T | 8% Necromone MUP |
| 6 | Formulation U | 4% Necromone MUP in EtOH |

The test substances were applied directly to prepared collagen membranes by pipetting 25 μL of the appropriate substance onto a membrane and spreading it evenly with the tip of the pipette.

Exposure Container.

A 30.5 cm×30.5 cm×30.5 cm rigid plastic frame supported by four, 4-cm high legs, with a sleeved entry on one side and a sliding door on the bottom.

Membrane Feeder.

Five wells (3 cm in diameter×8 mm in depth) in line on a hollow plastic block (6 cm wide×22 cm long×3 cm deep), which fits through the sliding door in the bottom of the exposure container. Hoses attached to each side of the block circulate heated water that is pumped from a water bath. The sliding door in the bottom of the exposure container covers and uncovers the wells in the membrane feeder, allowing mosquitoes to access the wells.

Test Set-Up.

The membrane feeder was connected to a heated water bath, and warm water passed through the feeder via a circulating pump so that the wells were warmed to 89-95° F. Seventy-two (72) mg of ATP (disodium salt) were added to 26 mL of warmed citrated bovine blood, which was poured into the wells until they were completely full.

The collagen membranes were briefly dipped in tap water and blotted with a paper towel then placed flat on a metal tray covered in wax paper (using vacuum grease as a temporary adhesive around the edges of the membranes). The membranes were then treated as described above, and left to age for two hours on a heating pad at 93-96° F. An additional membrane was prepared in the same way except that it received no treatment, in order to be used as a negative control. After the two hour aging interval, the membranes were placed over each of the wells (with vacuum grease as adhesive), completely covering the blood. Care was taken to eliminate all air bubbles from between the membrane and the surface of the blood.

Exposure to Mosquitoes.

The mosquitoes were released into the exposure container just prior to exposure to the membranes. The exposure container was then placed on the membrane feeder and the sliding door opened, allowing the mosquitoes to access the wells. The number of mosquitoes probing each membrane was recorded every two minutes for twenty minutes.

The above procedures were repeated until five replicates were completed. A new batch of 250 female mosquitoes and fresh blood were used for each replicate, and the wells were cleaned in between replicates. The position of the treatments was rotated for each replicate, so that each treatment was tested on each of the five wells.

A total of three tests were conducted, each assessing a combination of four of the formulations against an untreated membrane. Each formulation was assessed twice over the course of the three test days.

Statistical analyses.

Percent repellency was calculated for each replicate using the total number of probes with the following formula: Repellency=((C−T)/T)*100, where c=the total number of probes on the untreated well, and T=the total number of probes on the treated well.

Average percent repellency and standard error were then calculated across all five replicates.

TABLE 18
Repellency of six experimental formulations exposed to adult
female Aedes aegypti mosquitoes after being aged for 2 hours
(percent repellency based on comparison to untreated).

| | Average % repellency (SE) | | | |
|---|---|---|---|---|
| Formulation | | Test 1 | Test 2 | Test 3 |

| Formulation P | 99.2 | (0.8) | — | | 97.1 (2.9) |
|---|---|---|---|---|---|
| Formulation Q | 100 | (0) | 99.6 | (0.4) | — |
| Formulation R | 99.8 | (0.2) | 98.8 | (1.1) | — |

| | | | | |
|---|---|---|---|---|
| Formulation S | — | 100 | (0) | 99.2 (0.7) |
| Formulation T | — | 98.9 | (1.1) | 99.7 (0.3) |

| | | | | |
|---|---|---|---|---|
| Formulation U | 81.3 | (10.5) | — | 95.6 (1.9) |

### Example 28

An in vitro laboratory trial was conducted to evaluate the repellency of three experimental formulations (Formulation J, Formulation K, and Formulation L), aged for two and/or six hours after application, against female mosquitoes. Testing was conducted over three test days: two days to assess the two-hour aging interval, and one day to assess the six-hour aging interval. Two positive controls (20% DEET and Off! Botanicals, repellant) and an untreated (negative) control were also assessed on each test day for comparative purposes.

TABLE 19

| Formulation | Active ingredients |
|---|---|
| Off! Botanicals ® repellant | 10% p-menthane-3,8-diol |
| DEET in ethanol | 20% DEET |

Test substances were applied to prepared collagen membranes and aged for the appropriate time interval on a heating pad. The membranes were then placed over heated, blood-filled wells and exposed to mosquitoes. The number of mosquitoes probing each well was recorded at two minute intervals up to 20 minutes.

Results for Aedes aegypti:

Treatments Formulation K and Formulation L each provided consistently high repellency across both time intervals (99.4-100% and 100%, respectively). Formulation J demonstrated significantly lower repellency than most other treatments at the two hour aging intervals (66.4-70.1%). Results are summarized in Table 20 below.

TABLE 20
Repellency of three experimental formulations and two positive controls
exposed to adult female Aedes aegypti after being aged for two
and/or six hours (average percent and standard errors, n = 5).
Percent repellency calculated by comparison to untreated control.

| | Percent repellency (SB) | | | | | |
|---|---|---|---|---|---|---|
| | Aged | | Aged | | Aged | |
| Formulation | 2 Hours | | 2 Hours | | 6 Hours | |
| Formulation J | 66.4 | (13.3) | 70.1 | (4.7) | —* | |
| Formulation K | 100.0 | (0.0) | —* | | 99.4 | (0.6) |
| Formulation L | —* | | 100.0 | (0.0) | 100.0 | (0.0) |
| 20% DEET | 99.9 | (0.1) | 96.1 | (1.9) | 89.0 | (3.8) |
| Off! Botanicals ® repellant | 90.6 | (3.5) | 92.5 | (4.3) | 98.7 | (0.7) |

*Formulation was not tested.

Results for Anopheles quadrimaculatus: Treatments Formulation K and Formulation L each provided satisfactory repellency across both time intervals (83.6-95.8% and 87.7-91.4%, respectively) and provided similar efficacy to 20% DEET at two hours and Off! Botanicals® repellant at both ageing intervals. At six hours the experimental treatments provided superior efficacy compared to 20% DEET (75%). Results are summarized in Table 21 below.

TABLE 21
Repellency of two experimental formulations and two postive
controls exposed to adult female Anopheles quadrimaculatus
after being aged for two and six hours (average percent
and standard errors, n = 5). Percent repellency
calculated by comparison to untreated control.

Average % repellency (SE)

| Formulation | 2 Hours | 6 Hours | |
|---|---|---|---|
| Formulation K | 95.8 (1.9) | 83.6 | (8.6) |
| Formulation L | 87.7 (4.4) | 91.4 | (5.7) |
| 20% DEET | 96.8 (1.7) | 75.0 | (10.5) |
| Off! Botanicals ® repellant | 96.6 (2.1) | 92.9 | (4.4) |

Results for Culex quinquefasciatus:

Treatments Formulation K and Formulation L each provided consistently high repellency across both time intervals (96.9-100% and 99.5-100%, respectively) and provided similar efficacy to both 20% DEET and Off! Botanicals, repellant. Results are summarized in Table 22 below.

TABLE 22
Repellency of two experimental formulations and two postive controls
exposed to adult female Culex quinquefasciatus after being aged
for two and six hours (average percent and standard errors, n =
5). Percent repellency calculated by comparison to untreated control.

| | Average % repellency (SE) | | |
|---|---|---|---|
| Formulation | | 2 Hours | 6 Hours |

| | | | |
|---|---|---|---|
| Formulation K | 100.0 | (0.0) | 96.9 (2.2) |
| Formulation L | 100.0 | (0.0) | 99.5 (0.5) |
| 20% DEET | 91.1 | (5.8) | 98.2 (1.1) |
| Off! Botanicals ® repellant | 96.5 | (1.9) | 90.1 (8.2) |

In conclusion, treatments Formulation K and Formulation L were effective at repelling mosquitoes even when aged for up to six hours.

The foregoing should only be considered as illustrative of the principles of the invention. Further, since numerous modifications and changes may readily occur to those skilled in the art, it is not desired to limit the invention to the exact compositions of components and materials as described herein, and accordingly, all suitable modifications and equivalents may be resorted to, falling within the scope of the claimed invention.

What is claimed is:

1. An insect- or acarid-repellant composition comprising:
(a) a first active ingredient selected from the group consisting of dimethyl anthranilate, menthyl anthranilate, ethyl anthranilate, phenylethyl anthranilate and methyl anthranilate;
(b) a fatty acid;
(c) optionally at least one additional active ingredient; and
(d) a carrier;
wherein the ratio of first active ingredient to fatty acid is about 4:1 to about 5:1.

2. The composition of claim 1, wherein the fatty acid is selected from the group consisting of oleic acid, ricinoleic acid, linoleic acid palmitic acid, stearic acid, and any mixtures thereof.

3. The composition of claim 1, wherein the fatty acid is oleic acid.

4. The composition of claim 1 wherein the anthranilate ester is methyl anthranilate.

5. The composition of claim 1, wherein the fatty acid is oleic acid and the anthranilate ester is methyl anthranilate.

6. The composition of claim 1, further comprising an additional compound selected from the group consisting of a surfactant, emulsifying agent, a pH modifier, a thickening agent, an anti-oxidant, a preservative, a skin conditioning agent, an emollient, a humectant, a silicone moiety, an anti-inflammatory, a sun blocking agent, a topical anesthetic, vitamins, an antipruritic, an antibiotic, antifungal, an antiseptic, a vasoconstrictor, and any mixtures thereof.

7. The composition of claim 6, wherein the emulsifying agent is selected from the group consisting of polysaccharide ethers, polyglycosides, fatty acids, fatty alcohols, amine oxides, water-soluble cellulose, alkyl sulfonates, ethoxylated alkyl phenols, alkanolamides, betaines, zwitterionic surfactants, carboxylated alcohols, carboxylic acids, ethoxylated C₁₂-C₁₅ alcohols, polysorbates, behentrimonium methosulfate, isopropyl myristate, ethoxylated castor oil and any mixtures thereof.

8. The composition of claim 6, wherein the thickening agent is selected from the group consisting of carboxylic acid polymers, crosslinked acrylic acid with allyl ethers of sucrose, crosslinked polyacrylate polymers, polyacrylamide polymers, polysaccharides, gums and any mixtures thereof.

9. The composition of claim 8, wherein the polysaccharides are selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropylmethylcellulose, methyl hydroxyethylcellulose, microcrystalline cellulose, sodium cellulose sulfate, chitosan and any mixtures thereof.

10. The composition of claim 8, wherein the gums are selected from the group consisting of calcium carrageenan, carnitine, carrageenan, dextrin, gelatin, gellan gum, guar gum, guar hydroxypropyltrimonium chloride, hectorite, hydroxypropyl guar, karaya gum, kelp, locust bean gum, natto gum, potassium carrageenan, sclerotium gum, sodium carrageenan, tragacanth gum, xanthan gum and any mixtures thereof.

11. The composition of claim 1, wherein the carrier is a pharmaceutically suitable topical carrier and comprises water, glycerin, lecithin, a fatty acid or any combinations thereof.

12. The composition of claim 11, wherein the fatty acid is selected from the group consisting of oleic acid, ricinoleci acid, linoleic acid, palmitic acid, steric acid and any mixtures thereof.

13. The composition of claim 6, wherein the sun blocking agent is selected from the group consisting of PABA, PABA esters, butyl PABA, ethyl PABA, ethyl dihydroxypropyl PABA, benzophenones, avobenzone, cinnamates, salicylates anthranilates, ethyl urocanate, homosalate, dibenzoylmethanes, octocrylene, methylbenzylidene camphor, kaolin, talc, petrolatum, metal oxides and any mixtures thereof.

14. A method of repelling an insect from a subject comprising topically administering to a subject in need thereof the composition of claim 1.

15. The method of claim 14 wherein the subject is a mammalian subject.

16. The method of claim 14, wherein the subject is a human subject.

17. The method of claim 14, wherein the subject is a livestock animal.

18. The method of claim 15, wherein the composition is directly applied to the skin, the subject's apparel, and/or clothing.

19. A consumer care product comprising the composition of claim 1, wherein the ratio of the first active ingredient to fatty acid is about 5:1.

20. A moist towelette product comprising a woven or non-woven fabric or cellulosic substrate and the composition of claim 1.

21. A method for killing an insect or pest, comprising providing a composition of claim 1; and applying the composition to the insect or pest, whereby the insect or pest is killed, wherein the amount applied is sufficient to kill the insect or pest.

22. A method of repelling an insect or acarid from clothing or bedding, comprising deploying a composition of claim 1 to said clothing or bedding, whereby the insect is repelled when the insect comes into contact with the composition or vapors from the composition, wherein the composition is deployed by:
applying the composition topically to an article of clothing or bedding; or
laundering the article of clothing or bedding with a detergent or fabric softener or both that comprises the composition; or
drying the article of clothing or bedding with a fabric softener that comprises the composition.

23. A method of repelling an insect or acarid, comprising deploying an insect repellent composition of claim 1, whereby the insect or acarid is repelled when it comes into contact with the composition or vapors from the composition.

24. The method of claim 23, wherein the ratio of a first active ingredient to a fatty acid is about 5:1.

25. The method of claim 23, wherein the composition is deployed by:
applying the composition topically to an article of clothing of a human;
skin or hair of a human; or skin or fur of an animal; or
laundering an article of clothing of a human with a detergent or fabric softener or both that comprises the composition; or
drying an article of clothing of a human with a fabric softener that comprises the composition; or
applying the composition to bedding, bed boards, bed slats, a mattress, box springs, furniture, carpeting, baseboards or flooring or a combination thereof; or spraying the composition on to the surface of bedding, bed boards, bed slats, a mattress, box springs, furniture or carpeting; or
injecting the composition into the mattress, box springs, furniture or carpeting or a combination thereof; or
deploying an absorbent substrate or gel containing the composition in the vicinity of bed boards, bed slats, a mattress, box springs, furniture or carpeting so that vapors from the composition come into contact with a surface of the bed boards, bed slats, a mattress, box springs, furniture or carpeting; or injecting the composition into a wall space.

26. The composition of claim 1, wherein the composition is formulated as a personal care or cosmetic composition.

27. The composition of claim 26, wherein the personal care or cosmetic composition is formulated as a product selected from among insect repellents, skin care products, hair care products, and cleansing products.

28. The composition of claim 1 formulated as a household care composition.

29. The composition of claim 28, that is formulated as a product selected from among air deodorant/freshener compositions in liquid, gel or solid form, all purpose cleaner compositions, all purpose disinfectant compositions, deodorizing sprays and powders, dish detergents, fabric sizing compositions, fabric softening compositions, fabric static control compositions, hard surface cleanser compositions, hard surface detergents, hard surface sanitizing compositions, linen and bedding spray compositions, pesticide compositions, polishing compositions, laundry detergents, rug and upholstery shampoo compositions, cleaners and deodorizers, tile, toilet and tub cleaning and disinfectant compositions, waxes and cleaning compositions for treating wood floors or furniture, and waxes and cleaning compositions for automobiles.

30. The composition of claim 1, that is a fabric softening composition selected from among a liquid fabric softener, a fabric softening rinse, a fabric softening sheet, and a fabric softening gel.

31. A fabric treatment sheet comprising the composition of claim 1.

32. A method of treating a structure infested with insects or aracids, comprising deploying a composition of claim 1 onto said structure.

33. An insect- or acarid-repellant composition comprising:
(a) methyl anthranilate;
(b) oleic acid;
(c) optionally at least one additional active ingredient; and
(d) a carrier;
wherein the ratio of methyl anthranilate to oleic acid is about 1:10 to about 20:1.

34. The composition of claim 33 wherein the ratio of methyl anthranilate to oleic acid is about 4:1 to about 5:1.
